SEQUENCE LISTING (1 sequence)

SEQ ID NO: 1 - protein, 9 aa, Unknown
  source: /note=“Description of Unknown 2A C-terminal sequence”
  MOD_RES (5)..(5): Any amino acid
GDVEXNPGP

(19) United States
(12) Patent Application Publication
(10) Pub. No.: US 2024/0167055 A1
(43) Pub. Date: May 23, 2024

(54) COMPOSITIONS AND METHODS FOR PRODUCING AND CHARACTERIZING VIRAL VECTOR PRODUCER CELLS FOR CELL AND GENE THERAPY

(71) Applicant: iVexSol, Inc., Worcester, MA (US)

(72) Inventor: Michael GREENE, Groton, MA (US)

(21) Appl. No.: 18/281,146

(22) PCT Filed: Mar. 8, 2022
(86) PCT No.: PCT/US2022/019346

§ 371 Date: Sep. 8, 2023

Related U.S. Application Data
(60) Provisional application No. 63/158,841, filed on Mar. 9, 2021.

(51) Int. Cl. C12N 15/86

(52) U.S. Cl. CPC C12N 15/86; C12N 2740/16043; C12N 2740/16052

(57) ABSTRACT

The present disclosure provides compositions and methods for producing and characterizing stable viral vector producer cell lines that enable industrial scale production of viral vectors. Novel viral vector genome constructs, in which the constructs can be precisely mapped and viral vector genome constructs precisely quantified, are also disclosed for efficient production and characterization of viral vectors in mammalian cells.

### CROSS-REFERENCE TO RELATED APPLICATIONS

This application claims priority from U.S. Provisional Patent Application No. 63/158,841, filed Mar. 9, 2021, which is herein incorporated by reference in its entirety.

### SEQUENCE LISTING

The instant application contains a Sequence Listing which has been submitted electronically in ASCII format and is hereby incorporated by reference in its entirety. Said ASCII copy, created on Mar. 1, 2022, is named P35060WO00_SL.txt and is 616 bytes in size.

### FIELD

The present disclosure relates to the field of the production and characterization of viral vectors for cell and gene therapy.

### BACKGROUND

The growing number of gene therapy candidates combined with rapid progression through the clinical development has created a world-wide shortage of gene therapy vectors. More than 500 gene therapy and 100 cell therapy candidates are in different stages of development. Greater than 2200 clinical studies are ongoing across the globe. The strong and proven safety profile of viral vectors (e.g., lentiviral vectors) has underpinned a robust clinical development pipeline. However, the clinical manufacture and use of viral vectors, especially lentiviral vectors, also comes with several limitations. For example, conventional manufacturing methods and associated technologies are outdated and not scalable, provide low downstream process yields (˜20%), and require significant upfront capital and ongoing operational costs to establish. Furthermore, traditionally, viral vector manufacturing is seen as unpredictable and highly risky, resulting in demand greatly exceeding supply which in turn drives up prices. There is a need to identify new methods and improvement for manufacturing viral vectors by generating stable producer lines with high titer at high volumes.

### SUMMARY

In an aspect, the present disclosure provides a viral vector construct comprising a barcoded concatemer molecule.

In another aspect, the present disclosure provides a cell line comprising a viral vector construct comprising a barcoded concatemer molecule.

In another aspect, the present disclosure provides a method of making a viral vector construct comprising a barcoded concatemer molecule, wherein the method comprises ligating a unique molecular identifier to a viral vector genome construct to generate a barcoded viral vector genome construct, and ligating a barcoded viral vector genome construct to generate a barcoded concatemer molecule.

In another aspect, the present disclosure provides a method of making a stable viral vector producer cell line comprising a barcoded concatemer, wherein the method comprises introducing into a population of cells a viral vector genome construct, wherein the viral vector genome construct comprises a barcoded concatemer, comprising an expression cassette encoding one or more copies of a gene of interest (GOI) and one or more unique molecular identifiers, producing a population of transgenic cells comprising integrated sequences encoding a GOI and one or more unique molecular identifiers, selecting from a population of transgenic cells a cell clone producing a desired viral titer, and generating from a selected cell clone a stable viral vector producer cell line.

In a further aspect, the present disclosure provides a method of characterizing a viral vector comprising a barcoded concatemer molecule, wherein the viral vector is integrated, and wherein the barcoded concatemer comprises one or more unique molecular identifiers, and wherein the method comprises sequencing the barcoded concatemer molecule to generate sequence reads, assembling the generated sequence reads, and mapping the assembled sequence reads.

### BRIEF DESCRIPTION OF THE DRAWINGS

FIG. 1 provides an illustration of the genome organization of the HIV-1 Virus. The HIV-1 genome contains 9,749 bp. In addition to the gag, pol, and env genes common to all retroviruses, HIV-1 contains a regulatory gene—rev—that is indispensable for virus replication, and five accessory genes—tat, vif, vpr, vpu and nef—that, while dispensable for in vitro virus growth, are key for in vivo replication and pathogenesis. Further information about the biological functions of HIV-encoded proteins is provided in Table 1.

FIG. 2 provides an exemplary illustration of an exemplary concatemer molecule, and further depicts how ambiguous mapping can result during assembly of sequence reads generated from a repetitive molecule. FIG. 2A provides an exemplary illustration of a vector genome construct comprising a barcoded concatemer molecule. FIG. 2B provides an exemplary illustration of how sequencing an unbarcoded concatemer molecule results in reads that cannot be precisely mapped due to the repetitive elements.

FIG. 3 provides an exemplary illustration of an exemplary vector genome construct (VGC), prior to barcode ligation, flanked by restriction enzyme sites marked Enzyme A and Enzyme B. These exemplary restriction enzyme sites will be used to ligate a unique molecular identifier (a unique barcoded linker) to each VGC. When each VGC is linked to a unique molecular identifier (represented by different ‘oligonucleotide’ color configurations), and then further ligated to form a concatemer, each VGC junction within the concatemer will possess a unique oligonucleotide sequence, thereby enabling unambiguous sequence assembly and mapping.

FIG. 4 provides an exemplary illustration of an exemplary barcoded concatemer molecule. Unique molecular identifiers are depicted between each vector genome construct (VGC). Mapping sequence reads from a barcoded concatemer molecule results in unambiguous mapping, despite the repetitive elements inherent to a concatemer molecule, due to the unique sequences between every vector-vector junction and every vector-insert junction. Unambiguous mapping of a barcoded concatemer molecule enables derivation of a number of viral vector genomes within a given concatemer, as the number of junctions will be equivalent to the number of vector genomes.

FIG. 5 provides an illustration of a non-limiting example of a method of attaching barcoded linkers (unique molecular identifiers) to vector genome constructs (VGC). For example, VGC are restriction digested, to generate sticky ends, and subsequently purified. Separately, unique molecular identifiers, shown here as single-stranded DNA (ssDNA) oligomers, with 5′ biotinylated ends, for example, are mixed with streptavidin bound magnetic beads, for example. Unbound oligomers are washed away, to result in barcoded ssDNA oligomer-loaded beads. Sticky-end VGC are then mixed with oligomer-loaded beads. Following second strand synthesis and washing, all VGC without linkers are washed away. Restriction digestion, for example, can remove VGC with attached barcoded DNA oligomers from the magnetic beads. Further washing and purification steps may be performed prior to ligation to form a concatemer molecule.

FIG. 6 provides an illustration of a second non-limiting example of a method of attaching barcoded linkers (unique molecular identifiers) to vector genome constructs (VGC). For example, linkers are biotinylated, for example, restriction digested and purified, and subsequently mixed with magnetic beads that are coated, for example, with streptavidin. Separately, VGC are restriction digested and purified, and then mixed with barcoded linker-attached magnetic beads. Restriction digestion, for example, can remove VGC with attached barcoded linker from the magnetic beads. Further washing and purification steps may be performed prior to ligation to form a concatemer molecule.

FIG. 7 provides an illustration of a non-limiting example of a method of attaching barcoded linkers (unique molecular identifiers) to vector genome constructs (VGC) without the use of beads. For example, VGC and barcoded linkers are restriction digested, purified, mixed, and ligated. The resulting barcoded VGC can then be ligated to form a concatemer molecule.

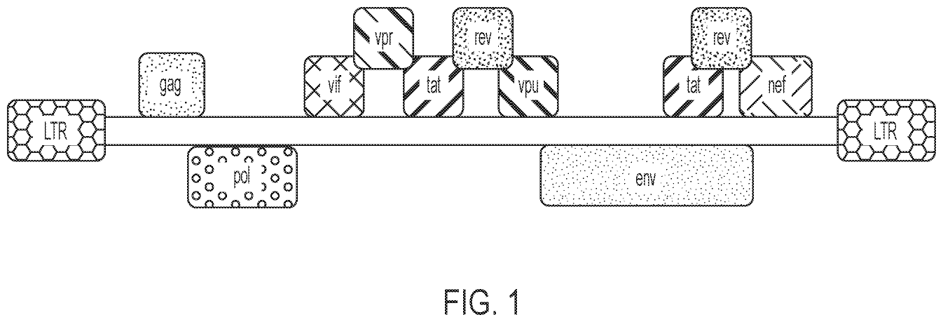

### DETAILED DESCRIPTION

Unless defined otherwise, technical and scientific terms used herein have the same meaning as commonly understood by one of ordinary skill in the art. One skilled in the art will recognize many methods can be used in the practice of the present disclosure. Indeed, the present disclosure is in no way limited to the methods and materials described. Where a term is provided in the singular, the inventors also contemplate aspects of the disclosure described by the plural of that term, and vice versa. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt. Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York).

Any references cited herein, including, e.g., all patents and publications are incorporated by reference in their entirety.

When a grouping of alternatives is presented, any and all combinations of the members that make up that grouping of alternatives is specifically envisioned. For example, if an item is selected from a group consisting of A, B. C, and D, the inventors specifically envision each alternative individually (e.g., A alone, B alone, etc.), as well as combinations such as A, B, and D; A and C; B and C; etc. The term “and/or” when used in a list of two or more items means any one of the listed items by itself or in combination with any one or more of the other listed items. For example, the expression “A and/or B” is intended to mean either or both of A and B—i.e., A alone, B alone, or A and B in combination. The expression “A, B and/or C” is intended to mean A alone, B alone, C alone, A and B in combination, A and C in combination, B and C in combination, or A, B, and C in combination.

When a range of numbers is provided herein, the range is understood to inclusive of the edges of the range as well as any number between the defined edges of the range. For example, “between 1 and 10” includes any number between 1 and 10, as well as the number 1 and the number 10.

As used herein, the singular form “a,” “an,” and “the” include plural references unless the context clearly dictates otherwise. For example, the term “a compound” or “at least one compound” may include a plurality of compounds, including mixtures thereof.

The term “about” is used herein to mean approximately, roughly, around, or in the region of. When the term “about” is used in conjunction with a numerical range, it modifies that range by extending the boundaries above and below the numerical values set forth and is understood to mean plus or minus 10%. For example, “about 100” would include from 90 to 110.

As used herein, the term “substantially”, when used to modify a quality, generally allows certain degree of variation without that quality being lost. For example, in certain aspects such degree of variation can be less than 0.1%, about 0.1%, about 0.2%, about 0.3%, about 0.4%, about 0.5%, about 0.6%, about 0.7%, about 0.8%, about 0.9%, about 1%, between 1-2%, between 2-3%, between 3-4%, between 4-5%, or greater than 5%.

To avoid any doubt, used herein, terms or phrases such as “about”, “at least”, “at least about”, “at most”. “less than”, “greater than”, “within” or “alike”, when followed by a series of list of numbers of percentages, such terms or phrases are deemed to modify each and every number of percentage in the series or list, regardless whether the adverb, preposition, or other modifier phrase is reproduced prior to each and every member.

As used herein, a “viral vector producer cell” refers to a cell which contains all the elements necessary for production of recombinant viral vector particles (including e.g., retroviral delivery systems). Typically, such viral vector producer cell contains one or more expression cassettes which are capable of expressing viral structural proteins (such as Gag, Pol and Env). A “stable viral vector producer cell” refers to a viral vector producer cell that contains in its nuclear genome, maintains episomally, or combination thereof, all the elements necessary for production of recombinant viral vector particles. A “stable viral vector producer cell line” refers a permanently established cell culture of stable viral vector producer cells that will proliferate indefinitely given appropriate fresh medium and space.

As used herein, a “recombinant viral vector” is an enveloped virion particle that contains an expressible polynucleotide sequence, and which is capable of penetrating a target host cell, thereby carrying the expressible sequence into the cell. In an aspect, an expressible is polynucleotide sequence comprises or encodes a gene of interest (GOI). The enveloped particle is preferably pseudotyped with an engineered or native viral envelope or capsid protein from another viral species, including lentiviruses or non-lentiviruses, which alters the host range and infectivity of a native virus.

As used herein, a “viral vector genome construct” is a construct which contains polynucleotide sequences which are packaged into a transducing recombinant viral vector. In an aspect, a viral vector genome construct, when comprising 5′ LTR and 3′ LTR and packaged with a functional integrase enzyme, can be used for the production of recombinant viral vectors that are capable of integrating into the host genome. In another aspect, a viral vector genome construct produces a recombinant viral vector comprising 5′ LTR and 3′ LTR and not capable of integrating into a host genome due to the lack of a functional integrase enzyme, which is also known as an integrase-defective lentiviral vector (IDLV).

As used herein, a “viral accessory construct” refers to a construct, plasmid or isolated nucleic acid molecule containing or encoding one or more elements that are useful for producing a functional recombinant viral vector in a compatible host cell, and packaging into it an expressible heterologous sequence.

As used herein, a “viral accessory protein” refers to a protein that is useful or necessary for producing a functional recombinant viral vector in a compatible host cell, and packaging into the viral vector an expressible heterologous sequence.

As used herein, a “viral vector construct” refers to either a viral vector genome construct or a viral accessory construct.

As used herein, the term “operably linked” describes the spatial relationship of two or more pieces of DNA such that one piece is capable of effecting an intended genetic outcome of another piece. For example, “operably linked” can denote a relationship between a regulatory region (typically a promoter element but may include an enhancer element) and the coding region of a gene, whereby the transcription of the coding region is under the control of the regulatory region.

As used herein, a “concatemer” is defined as a continuous DNA molecule that contains multiple copies of the same or substantially same DNA sequence linked in series. In an aspect, a concatemer may also contain one or more selection genes.

As used herein, a “barcoded concatemer” is defined as a continuous DNA molecule that contains one or more unique molecular identifiers interspersed between multiple copies of the same or substantially same DNA sequence linked in series. In an aspect, a barcoded concatemer may also contain one or more selection genes.

As used herein, a “unique molecular identifier” is an oligonucleotide barcode of a predetermined length that is used to distinguish between DNA molecules. In an aspect, a unique molecular identifier may comprise a linker containing a unique nucleic acid sequence (referred to herein as a barcode). A unique molecular identifier may be a single-stranded oligonucleotide or a double-stranded oligonucleotide.

As used herein, the term “ligation” refers to the formation of covalent phosphodiester linkages between two nucleic acid fragments via enzyme catalysis.

As used herein, the term “directional ligation” refers to ligation that occurs in a directed, specific, or fixed orientation.

As used herein, the term “sequential ligation” refers to ligation that occurs in an ordered sequence or step-wise in an ordered series of steps.

As used herein, the term “trans” refers to mechanisms acting from different molecules.

As used herein, the term “promoter” includes nucleic acid regions ranging in complexity and size from minimal promoters to promoters including upstream elements and enhancers.

As used herein, the term “transduction” refers to the delivery of a nucleic acid segment using a viral vector by means of viral vector.

As used herein, the term “transfection” refers to the introduction of foreign DNA into eukaryotic cells.

Without being bound to any theory, quality and quantity of infectious vector particles derived from a viral vector producer cell line is directly affected by the stoichiometric ratio of the lentiviral vector genomic RNA to the trans expressed accessory proteins. For any given lentiviral vector genome, the optimal ratio is not known a priori, and must be determined empirically through trial and error. As this biological fact is not often appreciated, the construction of stable cell lines has historically been accomplished by the addition of accessory genes one at a time in a serial fashion. This has assured progeny clones that had and expressed the accessory protein but limited the ability of the ultimate cell line to produce vector for lentiviral vector genomes with suboptimal ratios. The solution offered to this problem is to add all of the accessory elements at once in such a manner as to encourage multiple introductions of each of the elements. This not only speeds the development time of any given producer clone by collapsing the accessory gene introductions from multiple rounds of subcloning to a single round, it also allows for the generation of a diverse set of clones, each with different ratios, such that when the clones are screened the likelihood that we can find a clone that produces vector of the desired quality and quantity is increased without having to know a priori what that ratio would be.

In an aspect, this disclosure provides a method to produce a viral vector producer cell with a barcoded concatemer molecule that enables characterization of a viral vector producer cell, such as, but not limited to, quantification of the number of viral vector genome constructs within a given concatemer and localizing within a cellular genome an integration site of an integrated barcoded concatemer. In an aspect, this application provides a stable lentiviral vector producer cell line.

In an aspect, a vector producer cell line is produced from a parental cell line derived from an immortalized human cell line. In another aspect, a vector producer cell line grows in defined media either with or without human/animal derived serum. In another aspect, a vector producer cell line grows in an adherent or suspension adapted manner.

### Recombinant Viral Vectors

This disclosure relates to the manufacturing and/or production of recombinant viral vectors (also known as recombinant viral particles). The present disclosure relates to recombinant viral vectors, and constructs for their manufacture, which can be utilized to introduce expressible polynucleotide sequences of interest into host cells.

In an aspect, a viral vector producer cell disclosed herein comprises a retroviral production system, wherein the viral vector is derived from a retrovirus. Retroviruses comprise a family of enveloped viruses with a 7-12 kb single-stranded (ss) positive sense RNA genome. The retrovirus family includes five groups of oncogenic retroviruses, lentiviruses and spumaviruses.

Retroviral vector production systems typically involve separation of viral genome from viral packaging functions. Viral accessory proteins or viral accessory protein domains may be introduced via separate expression cassettes, or in trans. In an aspect, a viral accessory construct encodes or provides one or more viral accessory proteins involved in viral packaging.

In an aspect, the present disclosure relates to lentiviral vectors, and constructs for their manufacture, which can be utilized to introduce expressible polynucleotide sequences of interest into host cells. In an aspect, a lentiviral vector is an enveloped virion particle that contains an expressible polynucleotide sequence, and which is capable of penetrating a target host cell, thereby carrying the expressible sequence into the cell. The enveloped particle is preferably pseudotyped with an engineered or native viral envelope protein from another viral species, including non-lentiviruses, which alters the host range and infectivity of the native lentivirus.

Viral vectors described here can be utilized in a wide range of applications, including, e.g., for protein production (including vaccine production), for gene therapy (including gene replacement, gene editing, and synthetic biology), to deliver therapeutic polypeptides, to deliver siRNA, ribozymes, anti-sense, and other functional polynucleotides, etc. Such transduction vectors have the ability to carry single or dual genes, and to include inhibitory sequences (e.g., RNAi or antisense). In certain aspects, the transduction vector also carries a nucleic acid which comprises a modified 3′ LTR having reduced, but not absent, transcriptional activity.

Lentivirus is a group of retroviruses characterized by a long incubation period. They are classified into five serogroups according to the vertebrate hosts they infect: bovine, equine, feline, ovine/caprine and primate. Some examples of lentiviruses are human (HIV), simian (SIV) and feline (FIV) immunodeficiency viruses.

Lentiviruses can deliver large amounts of genetic information into the DNA of host cells and can integrate in both dividing and non-dividing cells. The viral genome is passed onto daughter cells during division, making it one of the most efficient gene delivery vectors.

The structure of HIV is different from that of other retroviruses. HIV is roughly spherical with a diameter of ˜120 nm. HIV is composed of two copies of positive single-stranded RNA that encode nine genes enclosed by a conical capsid containing 200) copies of the p24 protein. The ssRNA is tightly bound to nucleocapsid proteins, p7, and enzymes needed for the development of the virion: Reverse transcriptase (RT), Protease (PR). Ribonuclease and Integrase (IN). A matrix composed of p17 surrounds the capsid ensuring the integrity of the virion. This, in turn, is surrounded by an envelope composed of two layers of phospholipids taken from the membrane of a human cell when a newly formed virus particle buds from the cell. Embedded in the viral envelope are proteins from the host cell and about 70 copies of a complex HIV protein, known as Env, that protrudes through the surface of the virus particle. Env consists of a cap comprised of three gp120 molecules, and a stem consisting of three gp41 molecules that anchor the structure into the viral envelope. The glycoprotein complex enables the virus to attach and subsequently fuse with target cells to initiate the infectious cycle. Further information about the biological functions of each of the HIV-encoded proteins is provided in Table 1.

TABLE 1
Summary of the biological functions of HIV-encoded proteins.

| | Gene | | Precursor proteins → products |
|---|---|---|---|
| Essential Genes for | gag | Group-specific antigen | gag → MA, CA, SP1, NC, SP2, P6 |
| Vectorized | pol | Polymerase | pol → RT, RNase H, IN, PR |
| Lentivirus | env | Envelope | gp160 → gp120, gp41 |
| | rev | Regulator of expression | Important for major viral protein synthesis |
| | | of virion proteins | and essential for viral replication |
| Additional Genes | tat | HIV transactivator | Positive transcription regulator |
| found in | vif | Viral infectivity | Required for infectivity in some cell types |
| Wild-Type HIV | vpr | Virus protein R | Nuclear import of pre-integration complex |
| | | | and host cell cycle arrest |
| | vpu | Virus protein U | Proteasomal degradation of CD44 and virion |
| | | | release from infected cells |
| | nef | Negative factor | Roles in apoptosis and virus infectivity |

In an aspect, a viral vector producer cell disclosed herein comprises a lentiviral vector production system, wherein the viral vector is derived from a lentivirus. A lentivirus is a group of retroviruses that causes slow, gradual disease. A lentiviral vector particle produced by the lentiviral vector production system disclosed herein will be capable of transducing slowly-dividing cells, whereas standard retroviruses (gamma retroviruses) can infect only mitotically active cells. “Slowly dividing” cell types may divide approximately once every three to four days.

In the production of lentiviral vectors, multiple plasmids are used, one encoding envelope proteins (env plasmid), one or more plasmids encoding viral accessory proteins, and one plasmid comprising a gene of interest expression cassette between a lentiviral 3′-LTR and a lentiviral 5′-LTR to facilitate integration of the encoded gene(s) of interest into the host genome.

In an aspect, a viral vector may be a hybrid viral vector. The term “hybrid” as used herein refers to a vector, or nucleic acid component of a vector, that contains both lentiviral sequences and non-lentiviral sequences.

In an aspect, a viral vector producer cell disclosed herein comprises a herpesvirus vector production system, wherein the viral vector is derived from a herpesvirus.

In an aspect, a viral vector producer cell disclosed herein comprises an adenoviral vector production system, wherein the viral vector is derived from an adenovirus. Adenovirus is a nonenveloped virus with a 36-kilobase double-stranded DNA genome. Adenovirus is an attractive gene delivery vehicle candidate for its ability to grow as a high-titer recombinant virus, large transgene capacity, and efficient transduction of dividing and non-dividing cells. More than 50 human and nonhuman serotypes of adenovirus have been found to mediate gene delivery to a wide range of tissues.

In an aspect, a viral vector producer cell disclosed herein comprises an adeno-associated viral vector production system, wherein the viral vector is derived from an adeno-associated virus. Adeno-associated virus (AAV) is a nonenveloped virus with a 4.7 kb single-stranded DNA genome. More than 100 serotypes of AAV have been isolated from human and nonhuman tissues.

In a further aspect, a recombinant viral vector disclosed herein is derived from a virus comprising a mosaic genome structure. In a further aspect, recombinant viral vectors disclosed herein are target-specific. In a further aspect, target-specific viral vectors are receptor-targeted. In a further aspect, target-specific viral vectors comprise recombinant antibody molecules. Methods to produce target-specific viral vectors are known in the art. In a further aspect, a recombinant vector is derived from a partially or fully synthetic nucleic acid sequence.

Recombinant viral vectors disclosed herein may have one or more selectable, traceable or otherwise detectable marker elements. In an aspect, a selectable element is a reporter gene. In a further aspect, a selectable element is an epitope tag. In a further aspect, a viral vector may contain both a reporter gene and an epitope tag. In an aspect, an epitope tag may be selected or detected by methods known in the art, including but not limited to chromatography, enzyme assays, fluorescence assays, and immunodetection assays. In an aspect, immunodetection assays may include, but are not limited to immunoblotting, immunofluorescence, immunocytochemistry, and enzyme-linked immunosorbent assay (ELISA).

In a further aspect, a reporter gene may be detected by methods to detect absorbance. Methods to detect absorbance are known in the art. In an aspect, a reporter gene may be detected by methods to detect fluorescence. Methods to detect fluorescence are known in the art. In a further aspect, a reporter gene may be detected by methods to detect luminescence. Methods to detect luminescence are known in the art. In an aspect, a selectable marker gene is an antibiotic resistance gene. In a further aspect, an antibiotic gene encodes neomycin resistance. In a further aspect, an antibiotic gene encodes puromycin resistance.

In a further aspect, traceable marker genes may include genes encoding fluorescent proteins. Methods to select fluorescent proteins with different chromophores are known in the art. In a further aspect, fluorescent proteins may be Green fluorescent protein (GFP) or variants thereof, including, but not limited to Ultramarine, Blue, and Cyan fluorescent proteins. In a further aspect, a variant of a fluorescent protein may be an optimized variant. Methods to optimize traits of fluorescent proteins are known in the art and include but are not limited to methods to improve chromophore maturation, folding kinetics, and thermostability, among other traits.

In an aspect, a recombinant viral vector may be self-inactivating. The terms “self-inactivating” refer to a vector which is modified, such that the modification reduces the ability of the vector to mobilize once it has integrated into the genome of a target or host cell. For example, the modification may include deletions in the 3′ long terminal repeat (LTR) region. SIN vectors possess safety advantages over non-SIN vectors for gene delivery applications.

In another aspect, a recombinant viral vector produced here is a self-inactivating lentiviral vector (SIN vectors). In a SIN vector, the deletion of lentiviral enhancer and promoter sequences from the 3′ LTR results in the generation of vectors which, on infection of target cells, are incapable of transcribing vector-length RNA. Because of this modification, integrated SIN vectors are incapable of further replication, thus reducing the likelihood of generating replication-competent viruses as well as the danger of inadvertently influencing transcription activity of nearby endogenous promoters.

In another aspect, a recombinant viral vector produced here is a conditional SIN vector. For example, in an exemplary conditional SIN vector, the 3′ LTR U3 transcription regulatory elements can be replaced with an inducible promoter (e.g., Tet-responsive element).

### Viral Vector Genome Construct

In an aspect, disclosed herein is a viral vector genome construct encoding a recombinant viral vector genome. As used herein, a “recombinant viral vector genome” refers to a viral genome sequence engineered to become replication incompetent while harboring one or more additional sequence of interest that is typically not present in a natural form of the corresponding virus. In an aspect, a viral vector genome construct encodes a gene of interest. In an aspect, a gene of interest is operably linked to a promoter.

In an aspect, a gene of interest may be a candidate gene which is of known or potential significance in the pathophysiology of a disease. In a further aspect, a gene of interest may have a known or potential therapeutic or diagnostic application. In an aspect, a gene of interest may comprise a coding region. In a further aspect, a gene of interest may comprise a partial coding region. A gene of interest can be obtained for insertion into the viral vectors disclosed herein through a variety of techniques known in the art.

In a further aspect, a viral vector genome construct disclosed herein comprises one or more selectable or detectable reporter element(s). In an aspect, a selectable or detectable element is a reporter. In a further aspect, a selectable or detectable reporter element is an epitope tag. In an aspect, a selectable or detectable reporter element may be selected or detected by methods known in the art including, but not limited to, luminescence, absorbance, fluorescence, antibiotics, antigen-antibody interactions, or a combination thereof.

In an aspect, a viral vector genome construct disclosed herein comprises one or more elements selected from the group consisting of a promoter, 5′ and 3′ long terminal repeats, a packaging signal, a central polypurine tract, and a polyadenylation sequence. In another aspect, a viral vector genome construct disclosed herein comprises all the elements in the preceding sentence. In another aspect, a viral vector genome construct disclosed herein does not comprise a promoter, a 5′ long terminal repeat, a 3′ long terminal repeat, a packaging signal, a central polypurine tract, or a polyadenylation sequence. In another aspect, a viral vector genome construct disclosed herein can be used to produce a viral-like particle. In a further aspect, a long terminal repeat is a self-inactivating long terminal repeat.

A viral vector genome construct of the disclosure disclosed herein may be in the form of a concatemer. In an aspect, a concatemer may contain one or more transcription factors. In a further aspect, a transcription factor may be a ligand-responsive transcription factor. In a further aspect, a concatemer may contain one or more antibiotic selection genes. Antibiotic selection genes are known in the art. In an aspect, a concatemer is made and used as described in Throm et al. Blood, 2009; 113(21):5104-10. For example, a stable viral producer cell line can contain fully SIN lentiviral genome and viral accessory constructs stably integrated into the genome by concatemeric array transfection. Such array can be obtained through the ligation of DNA fragments encoding the SIN lentiviral vector genome, with drug resistance and/or other selection/reporter cassettes included into the array. In a further aspect, a concatemer molecule disclosed herein may be a barcoded concatemer molecule.

### Barcoded Concatemer Molecule

In an aspect, a viral vector genome construct comprises a barcoded concatemer molecule. In an aspect, a barcoded concatemer molecule comprises one or more copies of an expression cassettes encoding a gene of interest (GOI) and one or more unique molecular identifiers. In a further aspect, a barcoded concatemer molecule comprises one or more expression cassettes encoding a transcription factor. In another aspect, a barcoded concatemer molecule comprises one or more expression cassettes encoding an antibiotic selection gene. In another aspect, a barcoded concatemer molecule comprises one or more insulator sequences.

In a further aspect, a barcoded concatemer molecule comprises one or more elements selected from the group consisting of a 5′ long terminal repeat, a 3′ long terminal repeat, a packaging signal, and a central polypurine tract. In another aspect, a barcoded concatemer molecule does not comprise a 5′ long terminal repeat, a 3′ long terminal repeat, a packaging signal, or a central polypurine tract. In an aspect, a 5′ long terminal repeat is chimeric.

In an aspect, one or more expression cassettes are monocistronic. In a further aspect, one or more expression cassettes are polycistronic.

In an aspect, a unique molecular identifier is linked to an expression cassette. In a further aspect, linkage of a unique molecular identifier to an expression cassette occurs via ligation. Methods of ligation are known in the art. In an aspect, ligation is catalyzed via a DNA ligase enzyme. Ligases are known in the art. Examples of ligases that are classified under Class 6 according to the Nomenclature Committee of the International Union of Biochemistry and Molecular Biology are listed in the following database located on the worldwide web at https://www.enzyme-database.org/downloads/ec6.pdf. In a further aspect, ligation is directional. Methods of directional ligation are known in the art, such that ligation of one or more oligonucleotide fragments occurs in a directed, specific, or fixed orientation. In a further aspect, ligation is sequential. Methods of sequential ligation are known in the art, such that ligation of one or more oligonucleotide fragments occurs in sequence.

In an aspect, one or more unique molecular identifiers comprises one or more spacers. In a further aspect, one or more unique molecular identifiers does not comprise a spacer.

In an aspect, one or more unique molecular identifiers comprise equivalent numbers of nucleotides. In a further aspect, one or more unique molecular identifiers comprise different numbers of nucleotides.

In an aspect, a unique molecular identifier is flanked by one or more restriction enzyme sites. Restriction enzyme recognition sites are known in the art. Restriction enzymes are listed in databases available on the worldwide web, for example: http://rebase.neb.com/rebase/rebase.html.

In an aspect, the present disclosure further provides for methods of generating restriction enzyme sites. In a further aspect, methods of generating restriction enzyme recognition sites are known in the art. Restriction enzyme site identification, or restriction enzyme site mapping, can be performed using computational tools, such as RestrictionMapper.

In an aspect, a unique molecular identifier comprises a unique oligonucleotide sequence. In a further aspect, a unique molecular identifier comprises a known oligonucleotide sequence. In an aspect, a unique oligonucleotide sequence comprises randomly incorporated oligonucleotides.

In an aspect, the present disclosure provides for a method of making a viral vector construct comprising a barcoded concatemer molecule, where the method comprises ligating a unique molecular identifier to a viral vector genome construct to generate a barcoded viral vector genome construct and ligating the barcoded viral vector genome construct to generate a barcoded concatemer molecule.

In a further aspect, a viral vector construct comprises one or more unique molecular identifiers. In a further aspect, a viral vector construct comprises about 1 unique molecular identifier. In a further aspect, a viral vector construct comprises about 5 unique molecular identifiers. In a further aspect, a viral vector construct comprises about 10 unique molecular identifiers. In a further aspect, a viral vector construct comprises about 20 unique molecular identifiers. In a further aspect, a viral vector construct comprises about 25 unique molecular identifiers. In a further aspect, a viral vector construct comprises about 30 unique molecular identifiers. In a further aspect, a viral vector construct comprises about 35 unique molecular identifiers. In a further aspect, a viral vector construct comprises about 40 unique molecular identifiers. In a further aspect, a viral vector construct comprises about 45 unique molecular identifiers. In a further aspect, a viral vector construct comprises about 50 unique molecular identifiers. In a further aspect, a viral vector construct comprises about 60 unique molecular identifiers. In a further aspect, a viral vector construct comprises about 80 unique molecular identifiers. In a further aspect, a viral vector construct comprises about 100 unique molecular identifiers. In a further aspect, a viral vector construct comprises between about 1 and about 10 unique molecular identifiers.

In a further aspect, a viral vector construct comprises between about 1 and about 25 unique molecular identifiers. In a further aspect, a viral vector construct comprises between about 1 and about 50 unique molecular identifiers. In a further aspect, a viral vector construct comprises between about 1 and about 100 unique molecular identifiers. In a further aspect, a viral vector construct comprises between about 10 and about 25 unique molecular identifiers. In a further aspect, a viral vector construct comprises between about 25 and about 50 unique molecular identifiers. In a further aspect, a viral vector construct comprises between about 25 and about 100 unique molecular identifiers. In a further aspect, a viral vector construct comprises between about 50 and about 100 unique molecular identifiers.

In a further aspect, a unique molecular identifier is flanked by one or more restriction enzyme sites. In a further aspect, one or more restriction enzyme sites are located at a 5′ end of a unique molecular identifier. In another aspect, one or more restriction enzyme sites are located at a 3′ end of a unique molecular identifier.

In an aspect, a unique molecular identifier is biotinylated. In a further aspect, a unique molecular identifier is biotinylated at a 5′ end. In a further aspect, a unique molecular identifier is biotinylated at a 3′ end. Methods of biotinylation are known in the art. In an aspect, biotinylation may be performed via chemical methods. In a further aspect, biotinylation may be performed via enzymatic methods.

In another aspect, a unique molecular identifier is added prior to concatemer ligation. In another aspect, ligation of a unique molecular identifier to a viral vector genome construct occurs via an intermediary. In an aspect, an intermediary includes, for example, beads. In a further aspect, a bead is a magnetic bead. In another aspect, magnetic beads used in ligation of a unique molecular identifier with a viral vector genome comprise one or more streptavidin molecules. Methods of streptavidin-bead conjugation are known in the art. In a further aspect, ligation of a unique molecular identifier to a viral vector genome construct occurs without the use of an intermediary.

In a further aspect, ligation of a unique molecular identifier to a viral vector genome construct occurs prior to viral vector genome ligation to form a concatemer.

In a further aspect, a unique molecular identifier comprises a single-stranded nucleic acid. In another aspect, a unique molecular identifier comprises a double-stranded nucleic acid.

In an aspect, the present disclosure provides a method of making a stable viral vector producer cell line comprising a barcoded concatemer, wherein the method comprises: a) introducing into a population of cells a viral vector genome construct, where the viral vector genome construct comprises a barcoded concatemer, comprising an expression cassette encoding one or more copies of a gene of interest (GOI) and one or more unique molecular identifiers; b) producing a population of transgenic cells comprising integrated sequences encoding a GOI and one or more unique molecular identifiers; c) selecting from a population of transgenic cells a cell clone producing a desired viral titer; and d) generating from a cell clone a stable viral vector producer cell line.

In a further aspect, the present disclosure provides a method of characterizing a viral vector comprising a barcoded concatemer molecule, wherein said viral vector is integrated, and wherein a barcoded concatemer comprises one or more unique molecular identifiers, wherein the method comprises: a) sequencing the barcoded concatemer molecule to generate sequence reads; b) assembling the generated sequence reads; and c) mapping the assembled sequence reads.

In another aspect, the method of the present disclosure facilitates quantification of a number of viral vector genome constructs contained in a given barcoded concatemer. In a further aspect, quantification comprises quantifying a number of vector-vector junctions. In a further aspect, quantification comprises quantifying a number of vector-cellular genome junctions. In a further aspect, quantification comprises quantifying a number of vector-transgene junctions. In an aspect, a transgene is a gene of interest (GOI).

In a further aspect, the method of the present disclosure facilitates identification of a cellular genomic location of an integrated viral vector comprising a barcoded concatemer molecule.

### Viral Accessory Genes/Proteins/Constructs

In an aspect, a barcoded concatemer molecule can contain one or more viral accessory constructs.

In an aspect, a viral accessory construct encodes one or more accessory proteins including for example, structural proteins (e.g., the Gag precursor), processing proteins (e.g., the Pol precursor), and other proteins such as proteases, envelope protein. In another aspect, a viral accessary vector comprises sequences that provide the expression and regulatory signals needed to manufacture one or more accessory proteins in host cells and assemble functional viral particles. In one aspect, coding sequences for an Env, a Rev, and a Gag-Pol precursor are on the same plasmid or viral accessory construct. In another aspect, coding sequences for an Env, a Rev, and a Gag-Pol precursor are placed on separate plasmids or viral accessory constructs. In a further aspect, separate plasmids or viral accessory constructs are used for each coding sequence of the Gag. Pol, Rev, and Env proteins. In an aspect, a viral accessory construct may encode one or more structural and/or regulatory viral proteins, or functional fragments or domains thereof, selected from the group consisting of Group-specific antigen (Gag). RNA-dependent DNA polymerase (Pol), Regulator of expression of viral protein (Rev). Envelope (Env), Transactivator (Tat), Negative regulatory factor (Nef), Viral protein R (Vpr), Virus infectivity factor (Vif), Viral protein U (Vpu), and Viral protein X (Vpx). In another aspect, a functional fragment or domain can comprise one or more proteins selected from the group consisting of MA (Matrix [p17]), CA (Capsid [p24]), NC (Nucleocapsid [p9]), p6, Protease (p10), RT (p50), RNase H (p15), and Integrase (p31). In an aspect, coding sequences of one or more viral accessory proteins are operably linked. In an aspect, coding sequences of one or more viral accessory proteins are present on separate viral accessory constructs.

In an aspect, a viral accessory construct used here is for producing a recombinant lentiviral vector. In an aspect, a viral accessory construct used in the present disclosure can comprise one or more of the following elements, separately or collectively, in any suitable order or position, e.g., a) a heterologous promoter operably linked to a polynucleotide sequence coding for lentivirus Gag and Pol (e.g., a lentivirus Gag-Pol precursor); and b) a heterologous promoter operably linked to an env coding sequence.

Any suitable lentiviral 5′ LTR can be utilized in accordance with the present disclosure, including an LTR obtained from any lentivirus species, sub-species, strain or clade. This includes primate and non-primate lentiviruses. Specific examples of species, etc., include, but are not limited to, e.g., human immunodeficiency virus (HIV)-I (including subspecies, clades, or strains, such as A, B, C, D, E, F, and G. R5 and R5X4 viruses, etc.), HIV-2 (including subspecies, clades, or strains, such as, R5 and R5X4 viruses, etc.), simian immunodeficiency virus (SIV), simian-human immunodeficiency virus (SHIV), feline immunodeficiency virus (FIV), bovine immunodeficiency virus (BIV), caprine-arthritis-encephalitis virus, Jembrana disease virus, ovine lentivirus, visna/maedi virus, and equine infectious anemia virus.

Genomic reference sequences for such viruses are widely available, e.g., HIV-I (NC_001802), HIV-2 (NC_001722), SIV (NC_001549), SIV-2 (NC_004455), caprine arthritis-encephalitis virus (NC_001463), feline immunodeficiency virus (NC_001482), Jembrana disease virus (NC_001654), ovine lentivirus (NC_001511), visna/maedi virus (NC_001452), equine infectious anemia virus (NC_001450), and bovine immunodeficiency virus (NC_001413).

In an aspect, a lentiviral 5′ LTR used here comprises signals utilized in gene expression, including enhancer, promoter, transcription initiation (capping), transcription terminator, and poly adenylation. They are typically described as having U3, R, and U5 regions. The U3 region of the LTR contains enhancer, promoter and transcriptional regulatory signals, including RBEIII, NF-kB, SpI, AP-I and/or GABP motifs. The TATA box is located about 25 base pairs from the beginning of the R sequence, depending on the species and strain from which the 5′ LTR was obtained. A completely intact 5′ LTR can be utilized, or a modified copy can be utilized. Modifications preferably involve the R region, where a TAR sequence is substituted (see below), and/or deletion of all or part of a U5 region. The modified 5′ LTR preferably comprises promoter and enhancer activity, e.g., preferably native U3, modified R with a substituted TAR, and native U5.

In an aspect, a heterologous or non-viral promoter can be operably linked to a polynucleotide sequence coding for lentivirus Gag and Pol. By the term “operably linked,” it is meant that a promoter is positioned in such a way that it can drive transcription of the recited coding sequences. In an aspect, gag and pol coding sequences are organized as the gag-pol precursor in native lentivirus. The gag sequence codes for a 55-kD Gag precursor protein, also called p55. The p55 is cleaved by the virally encoded Protease 4 (a product of the pol gene) during the process of maturation into four smaller proteins designated MA (matrix [p17]), CA (capsid [p24]), NC (nucleocapsid [p9]), and p6. The Pol precursor protein is cleaved away from Gag by a virally encoded protease, and further digested to separate the Protease (p10). RT (p50), RNase H (p15), and Integrase (p31) activities.

In an aspect, one or more splice donor (SD) sites can be present in a viral vector genome construct or a viral accessory construct. A splice donor site is typically present between the 3′ end of the 5′LTR and the packaging sequence. A downstream splice acceptor (SA) can also be present, e.g., at the 3′ end of the pol sequences. The SD site can be present in multiple copies at any effective locations in the vector. The SD can have a native or mutated copy of a lentiviral sequence.

Native gag-pol sequences can be utilized in a viral accessory construct, or modifications can be made. These modifications can include, chimeric gag-pol, where the gag and pol sequences are obtained from different viruses (e.g., different species, subspecies, strains, clades, etc.), and/or where the sequences have been modified to improve transcription and/or translation, and/or reduce recombination. In other aspects of the present disclosure, the sequences coding for the Gag and Pol precursors (or parts thereof, e.g., one or more of MA (matrix [p17]), CA (capsid [p24]), NC (nucleocapsid [p9]), p6, protease (p10), RT (p50), RNase H (p15), and integrase (p31)) can be separated and placed on different vector constructs, where each sequence has its own expression signals.

The RNA genome of HIV-I contains an approximately 120 nucleotide psi-packaging signal that is recognized by the nucleocapsid (NC) domain of the Gag polyprotein during virus assembly. The critical portions of the packaging signal are between the major splice donor (SD) site and the gag initiation codon of the HIV provirus, about distal to the U5 region of the 5′ LTR. In an aspect, a packaging signal is functionally absent from the accessory construct to avoid packaging of functionally active Gag-Pol precursor into the viral transduction vector. See, e.g., U.S. Pat. No. 5,981,276 (Sodroski et al.), which describes vectors containing gag, but which lack the packaging signal.

Additional promoter and enhancer sequences can be placed upstream of the 5′ LTR in order to increase, improve, enhance, etc., transcription of the gag-pol precursor. Examples of useful promoters include, mammalian promoters (e.g., constitutive, inducible, tissue-specific), CMV. RSV, LTR from other lentiviral species, and other promoters as mentioned above and below. In addition, the construct can further comprise transcription termination signals, such as a polyA signal that is effective to terminate transcription driven by the promoter sequence. Any suitable poly A sequence can be utilized, e.g., sequences from beta globin (mammalian, human, rabbit, etc.), thymidine kinase, growth hormone, SV40, and many others.

In an aspect, gag-pol sequences are placed in opposite transcriptional orientations from the envelope sequences in a single viral accessory construct. By the latter, it is meant that the direction of transcription is opposite or reversed. This can be achieved by placing the corresponding promoters in opposite directions (i.e., facing each other) or using bi-directional promoters (e.g., Trinklein et al., Genome Research 2004, 14: 62-66). This arrangement can be utilized for safety purposes, e.g., to reduce the risk of recombination and/or the production of functional recombinant HIV genomes. Safety is increased with such vectors as there is no possibility that transcriptional read-through would result in an RNA that contains both functional gag-pol and env sequences. Transcriptional interference can be prevented by utilizing strong polyadenylation sequences that terminate transcription. Examples of strong transcription termination sequences are known in the art, including, e.g., rabbit beta-globin polyadenylation signal (Lanoix and Acheson, EMBO J. 1988 August; 7(8): 2515-22), See, also Plant et al., Molecular and Cellular Biology, April 2005, 25(8): 3276-3285. In addition, other elements can be inserted between the gag-pol and env coding sequences to facilitate transcriptional termination, such as a cis-acting ribozyme, or an RNAi sequence which are targeted to any putative read-through sequence. Similarly, instability sequences, termination sequences, and pause sites. e.g., can be placed between the coding sequences.

In an aspect, a viral accessory construct may encode structural viral proteins. In an aspect, a viral accessory construct may encode regulatory viral proteins. In an aspect, a viral accessory construct may encode both structural and regulatory viral proteins.

In an aspect, a viral accessory construct may encode structural and/or regulatory viral proteins that include, but are not limited to Group-specific antigen (Gag), RNA-dependent DNA polymerase (Pol), Regulator of expression of viral protein (Rev), Envelope (Env), Transactivator (Tat), Negative regulatory factor (Nef), Viral protein R (Vpr), Virus infectivity factor (Vif), Viral protein U (Vpu), and Viral protein X (Vpx).

Gag encodes structural proteins such as Matrix protein (MA), Capsid protein (CA), and Nucleocapsid protein (NC). Pol encodes proteins such as Protease (PR), Reverse transcriptase (RT), and Integrase (IN). Env encodes surface and transmembrane units of envelope protein.

In an aspect, encoded viral accessory proteins are fusion proteins. In an aspect, encoded viral accessory proteins are partial viral accessory proteins, such as protein domains. In an aspect, viral accessory protein domains may include, but are not limited to Capsid protein (CA), Matrix protein (MA), Nucleocapsid protein (NC), p6. Transcription factor specificity protein 1 (SP1), Reverse transcriptase (RT), Integrase (IN), Protease (PR), and Deoxyuridine triphosphatase (dUTPase or DU). In a further aspect, encoded viral accessory proteins include at least one full length protein or at least one protein domain.

In an aspect, a viral construct can further comprise an RRE element, including an RRE element which is obtained from a different lentiviral species than the 5′ LTR or gag and pol sequences. The RRE element is the binding site for the Rev polypeptide which is a 13-kD sequence-specific RNA binding protein. Constructs which contain the RRE sequence depend on the Rev polypeptide for efficient expression. Rev binds to a 240-base region of complex RNA secondary structure of the Rev response element (“RRE”) that is located within the second intron of HIV, distal to the pol and gag coding sequences. The binding of Rev to RRE facilitates the export of unspliced and incompletely spliced viral RNAs from the nucleus to the cytoplasm, thereby regulating the expression of HIV proteins. The RRE element can be in any suitable position on the construct, preferably following the Gag-Pol precursor in its approximate native position. Similarly, for the Tat polypeptide, any suitable Rev polypeptide can be utilized as long as it retains the ability to bind to RRE.

### Viral Capsids/Envelopes

Virus particles contain a viral genome packaged in a protein coat called the capsid. For some viruses, the capsid is surrounded by lipid bilayer that contains viral proteins, usually including the proteins that enable the virus to bind to the host cells. This lipid and protein structure is called the virus envelope and is derived from host cell membranes. The capsid and envelope play many roles in viral infection, including virus attachment to cells, entry into cells, release of the capsid contents into the cells, and packaging of newly formed viral particles. The capsid and envelope are also responsible for transfer of the viral genetic material from one cell to another. These structures also determine the stability characteristics of the virus particle, such as resistance to chemical or physical inactivation.

In an aspect, a stable viral vector producer cell line produces an envelope protein. In an aspect, envelope protein(s) employed in this cell line system use either the native HIV env gene (wild-type or codon optimized) or generate a pseudotyped particle using a biocompatible substitute including, but not limited to, amphotropic envelope protein, vesicular stomatitis vector (Indiana or other strain), measles or bioengineered chimeric measles envelope proteins, gibbon ape leukemia virus, or feline leukemia virus or bioengineered FLV chimeras.

In an aspect, viral vectors disclosed herein contain one or more capsid proteins. In an aspect, capsid proteins may be heterologous. Capsid proteins may be modified in order to alter vector biodistribution. In an aspect, capsid proteins may be genetically modified. In a further aspect, capsid proteins may be chemically modified. Strategies to genetically and chemically modify capsid proteins are known in the art.

In an aspect, viral vectors disclosed herein may have sequences encoding for one or more envelope (“Env”) proteins. Viral vector tropism is determined by the ability of the viral envelope protein to interact with molecules (proteins, lipids, or sugars) on the host cell.

In an aspect, a viral accessory construct can comprise an envelope module or expression cassette comprising a heterologous promoter operably linked to an envelope coding sequence. The Env polypeptide is displayed on the viral surface and is involved in the recognition and infection of host cells by a virus particle. The host range and specificity can be changed by modifying or substituting the envelope polypeptide, e.g., with an envelope expressed by a different (heterologous) viral species or which has otherwise been modified. This is called pseudotyping. See, e.g., Yee et al., Proc. Natl. Acad. Sci. USA 91: 9564-9568, 1994. Vesicular stomatitis virus protein G (VSV G) has been used extensively because of its broad species and tissue tropism and its ability to confer physical stability and high infectivity to vector particles. See, e.g., Yee el al., Methods Cell Biol., (1994) 43:99-112.

An envelope polypeptide can be utilized without limitation, including, e.g., HIV gp120 (including native and modified forms), Moloney murine leukemia virus (MoMuLV or MMLV), Harvey murine sarcoma virus (HaMuSV or HSV), murine mammary tumor virus (MuMTV or MMTV), gibbon ape leukemia virus (GALV), Rous sarcoma virus (RSV), hepatitis viruses, influenza viruses (VSV-G), Mokola virus, rabies, filovirus (e.g., Ebola and Marburg, such as GP1/GP2 envelope, including NP_066246 and Q05320), amphotropic, alphavirus, etc. Other examples include, e.g., envelope proteins from Togaviridae, Rhabdoviridae, Retroviridae, Poxviridae, Paramyxoviridae, and other enveloped virus families. Other example envelopes are derived from viruses listed in the following database located on the worldwide web at ncbi.nlm.nih.gov/genome/viruses.

Furthermore, a viral envelope protein can be modified or engineered to contain polypeptide sequences that allow the transduction vector to target and infect host cells outside its normal range or more specifically limit transduction to a cell or tissue type. For example, the envelope protein can be joined in-frame with targeting sequences, such as receptor ligands, antibodies (using an antigen-binding portion of an antibody or a recombinant antibody-type molecule, such as a single chain antibody), and polypeptide moieties or modifications thereof (e.g., where a glycosylation site is present in the targeting sequence) that, when displayed on the transduction vector coat, facilitate directed delivery of the virion particle to a target cell of interest. Furthermore, envelope proteins can further comprise sequences that modulate cell function. Modulating cell function with a transducing vector may increase or decrease transduction efficiency for certain cell types in a mixed population of cells. For example, stem cells could be transduced more specifically with envelope sequences containing ligands or binding partners that bind specifically to stem cells, rather than other cell types that are found in the blood or bone marrow. Such ligands are known in the art. Non-limiting examples are stem cell factor (SCF) and Flt-3 ligand. Other examples, include, e.g., antibodies (e.g., single-chain antibodies that are specific for a cell-type), and essentially any antigen (including receptors) that is specific for such tissues as lung, liver, pancreas, heart, endothelial, smooth muscle, breast, prostate, epithelial, etc.

Any heterologous promoter can be utilized to drive expression of a viral envelope coding sequence (or another viral accessory protein) when operably linked to it. Examples include, e.g., CMV, EF1 alpha, EF1 alpha-HTLV-1 hybrid promoter, ferritin promoters, inducible promoters, constitutive promoters, and other promoters mentioned herein, etc.

In an aspect, encoded envelope proteins are endogenous. In a further aspect, encoded envelope proteins are heterologous. Heterologous envelope proteins of the viral vectors disclosed herein may be generated using any envelope protein that is biocompatible. Biocompatibility can be determined using methods known in the art.

In an aspect, env may be derived from human immunodeficiency virus (HIV). In an aspect, a sequence encoding an HIV-derived envelope gene may be wild-type. In a further aspect, a sequence encoding an HIV-derived envelope gene may be codon-optimized.

Env may also be generated as a pseudotyped particle. Pseudotyping enables the engineering of viral vector particles with different target cell specificities, to expand and/or to alter the host range of the native virus from which the envelope protein was derived.

In an aspect, the viral vectors disclosed herein may be amphotropic pseudotyped viral vectors. In an aspect, the viral vectors disclosed herein may be ecotropic pseudotyped viral vectors. In an aspect, the viral vectors disclosed herein may be pantropic pseudotyped viral vectors. Envelope protein sequences encoded by the viral vectors disclosed herein may be derived from any species of the genera Vesiculovirus, Gammaretrovirus, or Morbillivirus.

In an aspect, envelope proteins may be derived from a species of the Vesiculovirus genus including, but not limited to, vesicular stomatitis New Jersey virus (VSV-NJ), and vesicular stomatitis Indiana virus (VSV-IN). In a further aspect, envelope proteins may be derived from any vesicular stomatitis virus serotype. In a further aspect, envelope proteins may be truncated proteins. In a further aspect, envelope proteins may be bioengineered chimeric vesiculovirus proteins.

In an aspect, envelope proteins may be derived from a species of the Gammaretrovirus genus, including, but not limited to gibbon ape leukemia virus (GALV) and feline leukemia virus (FLV). In a further aspect, envelope proteins may be bioengineered chimeric gammaretrovirus proteins, including GALV chimeras and FLV chimeras. A “chimera” as defined herein refers to a biological entity, such as a virus, that is composed of two or more genetic fragments of distinct origin or of distinct composition.

In an aspect, envelope proteins may be derived from a species of the Morbillivirus genus including, but not limited to, measles virus. In a further aspect, envelope proteins may be bioengineered chimeric morbillivirus proteins, including bioengineered chimeric measles envelope proteins. Methods of bioengineering chimeric envelope proteins are known in the art.

### Optional Tat

In an aspect, a stable viral vector producer cell line comprises or produces a Tat protein. In another aspect, a stable viral vector producer cell line does not produce a Tat protein. In the absence of a Tat protein, a lentiviral genome vector is modified such that the HIV promoter in the 5′ LTR is replaced with a heterologous enhancer/promoter to ensure transcription. In an aspect, such promoter could be either viral (like CMV) or cellular (like EF1-α).

In another aspect, a viral accessory construct can further comprise a TAR element that is obtained from a different lentiviral species, group, sub-species, sub-group, strain, or clade than the 5′ LTR and/or the gag and pol sequences that are present in it, i.e., it is heterologous to other lentiviral elements present in the construct. The TAR is preferably present in the 5′ LTR in its normal location, e.g., between the U3 and U5 elements of the LTR, e.g., where the native R is replaced by R′ of a heterologous lentiviral species.

The TAR element is a trans-activating response region or response element that is located in the 5′LTR (e.g., R) of the viral DNA and at the 5′ terminus of the corresponding RNA. When present in the lentiviral RNA, the transcriptional transactivator, Tat, binds to it, activating transcription from the HIV LTR many-fold. Tat is an RNA binding protein that binds to a short-stem loop structure formed by the TAR element.

When a heterologous TAR element is utilized, the 5′ LTR can be modified routinely by substituting its native TAR for a TAR sequence from another species. Examples of TAR regions are widely known. See, e.g., De Areliano et al., AIDS Res. Human Retro. 2005, 21: 949-954. Such a modified lentiviral 5′ LTR can comprise intact U3 and U5 regions, such that the LTR is completely functional. The TAR region or the entire R can be substituted.

As indicated above, the Tat polypeptide binds to the TAR sequence. The coding sequence for Tat can be present in a viral accessory construct. Any Tat polypeptide can be utilized as long as it is capable of binding to TAR and activating transcription of the RNA. This includes native Tat sequences which are obtained from the same or different species as the cognate TAR element, as well as engineered and modified Tat sequences.

### Promoters

In an aspect, a construct disclosed here contains one or more expression cassettes that express an accessory protein or RNA molecule under the control of a constitutive, inducible, switched, recombined, disrupted/edited promoter or promoter/enhancer. In an aspect, a promoter is a minimal promoter with upstream cis regulatory to determine spatio-temporal expression pattern of the promoter. Upstream regulatory elements may include cis-acting elements (or cis-acting motifs) or transcription factor binding sites. In a further aspect, the promoter comprises a combination of heterologous upstream regulatory elements.

In an aspect, a promoter is a promoter/enhancer. As used herein, the term promoter/enhancer refers to a segment of DNA that contains sequences capable of providing both promoter and enhancer functions. The promoter/enhancer may be endogenous or exogenous or heterologous. An endogenous promoter/enhancer is one which is naturally linked with a given gene in a native viral genome. An exogenous or heterologous enhancer/promoter is one which is placed in juxtaposition to a gene by means of molecular biology techniques such that the transcription of that gene is directed by the linked promoter/enhancer.

In an aspect, a promoter is an inducible promoter. In an aspect, an inducible promoter is positively inducible and regulated by positive control. In an aspect, an inducible promoter is negatively inducible, and regulated by negative control.

In a further aspect, an inducible promoter may be a chemically inducible promoter. Chemically inducible promoters are known in the art. In a further aspect, a chemically inducible promoter may be a tetracycline controllable promoter. In a further aspect, a tetracycline-controllable promoter is a natural promoter. In a further aspect, a tetracycline-controllable promoter is a synthetic promoter.

In a further aspect, an inducible promoter may be a temperature inducible promoter. In a further aspect, an inducible promoter may be alight inducible promoter. In a further aspect, an inducible promoter may be a physiologically regulated promoter.

In an aspect, a promoter may be a constitutive promoter. In an aspect, a promoter may be a switched promoter. In an aspect, a promoter may be a recombined promoter. In an aspect, a promoter may be a disrupted/edited promoter.

In an aspect, a promoter element may be naturally derivable. In a further aspect, a promoter may contain sequences derived from a eukaryotic promoter including, but not limited to CMV, EF1a, SV40, PGK1, Ubc, human beta actin, CAG, TRE, CaMKIIa, Cal1, 10, H1, and U6.

In a further aspect, a promoter comprises synthetic elements. Methods to prepare synthetic promoters are known in the art. In an aspect, a synthetic promoter is a constitutive synthetic promoter. In an aspect, a synthetic promoter is an inducible synthetic promoter. In an aspect, a synthetic promoter is a tissue specific synthetic promoter.

### Polyadenylation Sequences

In an aspect, a viral vector genome construct or a viral accessory construct comprises one or more polyadenylation sequences. Expression of recombinant DNA sequences in eukaryotic cells requires expression of signals to direct termination and polyadenylation of the resulting transcript. The term “polyadenylation sequence” as used herein refers to a nucleic acid sequence that directs the termination and polyadenylation of a nascent formed RNA transcript. Transcripts lacking a poly A tail may be unstable and quickly degraded. A poly A signal utilized in a viral vector genome construct disclosed herein may be heterologous or endogenous. An endogenous poly A signal refers to a poly A sequence that is found naturally at the 3′ end of the coding region of a given gene. A heterologous poly A signal refers to a poly A sequence that is isolated from one gene and placed at the 3′ end of another gene.

### Expression Cassettes

In an aspect, a viral vector genome construct and/or a viral accessory construct described here comprise one or more expression cassettes. Expression cassettes may be a monocistronic expression cassette or a polycistronic expression cassette.

In an aspect, a polycistronic expression cassette contains one or more viral skip sequences. Viral skip sequences are “self-cleaving” 2A peptides, which are 18-22 amino acid viral oligopeptides that mediate “cleavage” of polypeptides during translation in eukaryotic cells. The “2A” designation refers to a specific region of the viral genome. The mechanism of 2A cleavage is ribosome skipping, mediated by a highly conserved C-terminal sequences essential to the creation of steric hindrance. In an aspect, viral skip sequences may include 2A peptides derived from porcine teschovirus-1 2A (P2A). In an aspect, viral skip sequences may include 2A peptides derived from Thosea asigna virus 2A (T2A). In an aspect, viral skip sequences may include 2A peptides derived from equine rhinitis A virus (E2A). In an aspect, viral skip sequences may include 2A peptides derived from foot-and-mouth disease virus (F2A). In a further aspect, viral skip sequences may be derived from any virus with a 2A sequence substantially similar to the conserved “2A” C-terminal sequence GDVEXNPGP (SEQ ID NO: 1).

In an aspect, a polycistronic expression cassette contains one or more internal ribosome entry site elements (IRES). An IRES element is a cis-acting RNA region that promotes internal initiation of protein synthesis. An IRES sequence is recognized by a ribosome and can therefore be used to drive translation of multiple proteins off a single transcript.

In a further aspect, a polycistronic expression cassette contains one or more viral skip sequences and one or more internal ribosome entry site elements.

In an aspect, polycistronic expression cassettes encode for sequences that provide a similar mechanism to viral skip sequences or internal ribosome entry sequences.

### Codon Optimization

Expression cassettes contain sequences that encode one or more viral accessory proteins. In an aspect, a viral accessory protein may be encoded by a wild-type sequence. In an aspect, a viral accessory protein may be encoded by a mutated sequence. In a further aspect, a viral Integrase is encoded by a mutated sequence. In a further aspect, a viral accessory protein may be encoded by a codon optimized sequence. Codon optimization is commonly used to increase production of recombinant proteins or viral vectors. Codon optimization is a desirable molecular tool to address codon usage bias. Codon usage bias is a feature of all genomes and reflects the frequency of codon distribution within a genome is referred to as codon usage bias. Codon usage is variable between species, and preferred codons are more frequently used in highly expressed genes. Transfer RNAs, or tRNAs, reflect the codon usage in a given organism, and therefore the abundance of particular tRNAs is variable between organisms. Codon optimization is a process by which DNA sequences are modified by introducing silent mutations to generate synonymous codons.

In a further aspect, an expression cassette may contain sequences that are all wild-type sequences, all mutated sequences, all codon optimized sequences, or a combination of wild type, mutated, and codon optimized sequences. In an aspect, expression of Rev, Tat, Nef, Vpr, Vif, Vpu/Vpx when included, is from wild-type or codon optimized constructs which are polycistronic using viral skip sequences (such as P2A, or T2A) or internal ribosome entry sequences or other similar mechanisms, or as a single message per transcript. In an aspect, expression of Gag-Pol is from a wild-type or codon optimized polycistronic message, or as separate gag and pol constructs, or as further separated CA, MA SP1. NC, p6, RT, IN, PR, and/or DU constructs.

### Introducing Viral Vectors to Target or Host Cells

In an aspect, the introduction of one or more constructs into a cell is achieved using a standard chemical, biological, or physical methods including, but not limited to, lipofectamine or lipofectamine-like chemical reagents, polyethyleneimine (PET), calcium phosphate crystals, retroviral vector, lentiviral vector, nanoparticles or nanoparticle-like reagents, or electroporation. In another aspect, incorporation of these constructs into the cell line genome is achieved using biological recombinant enzymes including, but not limited to, integrase, transposase, recombinase, the CRISPR-Cas9 system, or utilizing spontaneous or targeted insertion using cellular DNA repair machinery.

In an aspect, methods of introducing viral vector constructs to a target or host cell may include transduction or transfection. Transfection and transduction may be performed using a variety of techniques known in the art and may include optimizations for enhancing transfection or transduction efficiency. In an aspect, optimization may comprise freeze-thawing reagents.

In an aspect, viral vector constructs are introduced to target or host cells using chemical methods known in the art. In an aspect, viral vector constructs are introduced to target or host cells using biological methods known in the art. In an aspect, viral vector constructs are introduced to target or host cells using physical methods known in the art.

In an aspect, viral vector constructs may be introduced to a target or host cell by methods comprising optical techniques. In an aspect, viral vector constructs may be introduced to a target or host cell by methods comprising magnetic techniques. In an aspect, viral vector constructs may be introduced to a target or host cell by methods comprising biolistic techniques. In an aspect, viral vector constructs may be introduced to a target or host cell by methods comprising polymer-based techniques. In an aspect, viral vector constructs may be introduced to a target or host cell by methods comprising liposome-based techniques. In an aspect, viral vector constructs may be introduced to a target or host cell by methods comprising nanoparticle-based techniques. In a further aspect, viral vector constructs may be introduced to a target or host cell by a combination of methods comprising a combination of techniques including, but not limited to optical, magnetic, biolistic, polymer-based, liposome-based, and nanoparticle-based techniques.

In a further aspect, viral vector constructs may be introduced to a target or host cell by methods comprising electroporation. In a further aspect, viral vector constructs may be introduced to a target or host cell by methods comprising sonoporation. In a further aspect, viral vector constructs may be introduced to a target or host cell by methods comprising mechanoporation. In a further aspect, viral vector constructs may be introduced to a target or host cell by methods comprising photoporation.

In a further aspect, methods of introduction may also comprise methods that involve use of a cationic polymer, calcium phosphate, cationic lipid, or a combination thereof. In an aspect, a cationic polymer is hexadimethrine bromide (commercial brand name Polybrene).

In a further aspect, methods of introduction may also comprise methods that involve use of a retrovirus, lentivirus, transposon, transcription activator-like effector nuclease (TALEN), Zinc Finger nuclease, meganuclease, transposase, a CRISPR-related nuclease (e.g., CRISPR/Cas9, Cas12a, etc.), or recombinase. In an aspect, a recombinase may be a Cre-recombinase, Flippase recombinase, or a derivative thereof.

Methods to promote the integration of nucleic acids into production cells are known in the art, and can include, but are not limited to, linearizing a nucleic acid construct.

In an aspect, one or more viral vector constructs may be stably integrated or episomally maintained within the viral vector production cell. Gene expression of sequences encoded by any of the introduced viral vectors may occur from integrated sequences or episomes.

In an aspect, a viral vector production cell stably expressing some of the components may be transfected with remaining components that are required for vector production. Transfection of the remaining components required for viral vector production may be transient.

A viral vector construct may integrate randomly or in a site-specific manner upon introduction into a host or target cell.

### Viral Vector Production Cells

The disclosure disclosed herein provides a method of making viral vector particles in vitro by introducing one or more viral vector constructs of the disclosure into a compatible target cell or host cell and growing the cell under conditions which result in cell expansion and expression of the vector components. The terms “target cell” and “host cell” as used herein are interchangeable.

A viral vector production cell is a target cell or host cell that is capable of producing a viral vector or viral vector particle upon introduction of one or more viral vector constructs.

In an aspect, a viral vector production cell is a transgenic cell. As used herein, the term “transgenic cell” refers to a cell comprising genetic material that has been transferred naturally or by any of a number of genetic engineering techniques known in the art from one cell type to another cell type. In a further aspect, a transgenic cell refers to a cell comprising experimentally constructed genetic material. In an aspect, a viral vector production cell population is polyclonal. Polyclonal cells comprise a heterogeneous population of cells with multiple clones that may have variations in the number of integration events and sites of integration across the cells. In a further aspect, a viral vector production cell population is monoclonal.

In an aspect, a viral vector production cell is from a cell line that has been expanded from a selected viral vector production cell clone.

Viral vector production cell clones may be derived from a polyclonal population by methods known in the art. Methods of selection include, but are not limited to, limiting dilution, single cell sorting, single cell selection, and combinations thereof. Limiting dilution may be performed by methods known in the art. Single cell sorting may be performed by methods known in the art, including, but not limited to, single cell printing, fluorescence activated cell sorting (FACS), and magnetic activated cell sorting. Single cell selection may be performed by selection methods known in the art, including, but not limited to selection for an epitope, a protein, a reporter gene, or combination thereof. In a further aspect, single cell selection methods may comprise selection via one or more metabolic or antibiotic properties.

In an aspect, viral vector production cell clones or cell lines grow in an adherent manner. In an aspect, viral vector production cell clones or cell lines grow in suspension. In a further aspect, viral vector production cell clones or cell lines that grow in an adherent manner may be suspension-adapted. In a further aspect, viral vector production cell clones or cell lines that grow in an adherent form.

In an aspect, viral vector production cell clones or cell lines are cultured in serum-supplemented or serum-free media. A person of skill in the art will be able to select an appropriate media for the given viral vector production cell type, and to modify the media composition at various stages of the method disclosed herein. Media may have a selection of secreted cellular proteins, diffusible nutrients, amino acids, organic salts, inorganic salts, vitamins, trace metals, sugars, and other growth-promoting substances such as cytokines. Media may be supplemented with glutamine or an alternative thereof.

In an aspect, viral vector production cell clones or cell lines may be any eukaryotic cell that supports the lifecycle of the specific virus from which the vector is derived. In an aspect, for a retroviral vector, a production cell clones or cell lines may be any eukaryotic cell that supports a retrovirus life cycle. In an aspect, for a lentiviral vector, a production cell clones or cell lines may be any eukaryotic cell that supports a lentivirus life cycle. In an aspect, for a herpesvirus vector, a production cell clones or cell lines may be any eukaryotic cell that supports a herpesvirus life cycle. In an aspect, for an adenoviral vector, a production cell clones or cell lines may be any eukaryotic cell that supports an adenovirus life cycle. In an aspect, for an adeno-associated viral vector, a production cell clones or cell lines may be any eukaryotic cell that supports an adeno-associated virus life cycle.

In an aspect, viral vector production cell clones or cell lines are immortalized. Cell lines may be commercially available or non-commercially available laboratory-derivatives. In a further aspect, viral vector production cell clones or cell lines are of eukaryotic origin. In an aspect, viral vector production cell clones or cell lines are of mammalian origin. Mammalian cells for the production of viral vectors are known in the art. In an aspect, viral vector production cell clones or cell lines are of human origin.

In a further aspect, a viral vector producer cell line is developed in or from human embryonic kidney (HEK) 293 cells, which are highly transfectable. In a further aspect, a viral vector producer cell line is a derivate of HEK293 cells, such as HEK293T or HEK293F cells. In a further aspect, cell types for viral vector production cell clones or cell lines include, but are not limited to, HeLa cells, Vero cells, Chinese Hamster Ovary (CHO) cells, A549 cells, and NIH 3T3 cells.

### Characterization of Produced Viral Vector

Viral vector particles produced by a viral vector producer cell clone or cell line may be characterized by a variety of methods known to those of skill in the art.

In an aspect, a viral vector particle produced by a method disclosed herein is a psuedotyped viral particle. Pseudotyped viral particles may be produced by substituting viral attachment proteins from one viral serotype with another. As used herein, a “viral attachment protein” refers to a viral capsid protein or a viral envelope protein.

In an aspect, a viral vector particle produced by a method disclosed herein is a mosaic viral particle. Mosaic viral particles may be produced by mixing different viral attachment proteins from different viral variants.

In an aspect, a viral vector particle produced by a method disclosed herein is a chimeric viral particle. Chimeric viral particles may be produced by methods that include swapping smaller domains of viral attachment proteins between serotypes (via rational methods or high throughput recombination techniques).

From a stable viral vector producing cell clone or cell line, viral vector genome and accessory proteins may be characterized quantitatively or qualitatively. In an aspect, the stoichiometric ratio of viral vector genome and one or more accessory proteins may be determined. In a further aspect, the level of viral vector genome and one or more accessory proteins may be determined.

An integration profile of a selected cell clone or cell line may be determined. In an aspect, an integration profile or an insertional profile may be detected by methods known in the art such as inverse PCR, linear amplification-mediated PCR or ligation-mediated PCR. Vector flanking sequences detected by such methods can then be mapped to a host cell genome and compared to a reference set. Mapping can be performed using computational tools to map and analyze vector-flanking sequences, such as QuickMap.

In an aspect, recombinant viral vectors may be harvested from a cell clone or a cell line. In an aspect, the cell line is monoclonal. Harvested viral vectors may be characterized qualitatively or quantitatively. In an aspect, viral titer is expressed in transducing units per milliliter (t.u./ml).

Viral titer may be determined using physical or functional titration. In an aspect, titration methods include but are not limited to transduction of indicator cells using dose-dependent quantities of vector supernatant.

In a further aspect, viral titer is determined by assaying for viral nucleic acid or viral protein. In an aspect, viral nucleic acid can be detected using polymerase chain reaction (PCR), reverse transcription PCR (RT-PCR), dot blot hybridization, Southern blot hybridization, or northern blot hybridization. In a further aspect, viral protection can be detected via an immunoassay. Immunoassays include but are not limited to immunoblotting (Western blotting), immunofluorescence, immunocytochemistry, and enzyme-linked immunosorbent assay (ELISA).

In a further aspect, transduced indicator cells may be assessed using PCR. Quantification by PCR may be performed using relative quantification or absolute quantification. Methods for relative or absolute quantification by PCR are known in the art.

In a further aspect, methods of viral titer determination are enzyme immunoassays. Harvested viral particles may be quantified by measuring the amount of a viral capsid protein using immunoassays specific to the virus from which the viral capsid protein was derived (for example, p24 for HIV).

Viral vector particles produced by methods disclosed herein may be concentrated and/or purified using flow-through ultracentrifugation and high-speed centrifugation, and tangential flow filtration. Flow through ultracentrifugation has been used in the past for the purification of RNA tumor viruses (Toplin et al., Applied Microbiology 15: 582-589, 1967; Burger et al., Journal of the National Cancer Institute 45: 499-503, 1970). The present disclosure provides the use of flow-through ultracentrifugation for the purification of lentiviral vectors. This method can comprise one or more of the following steps. For example, a lentiviral vector can be produced from cells using a cell factory or bioreactor system. A transient transfection system (see above) can be used or packaging or producer cell lines can also similarly be used. A pre-clarification step prior to loading the material into the ultracentrifuge could be used if desired. Flow-through ultracentrifugation can be performed using continuous flow or batch sedimentation. The materials used for sedimentation are, e.g.: Cesium chloride (CsCl), potassium tartrate and potassium bromide, which create high densities with low viscosity although they are all corrosive. CsCl is frequently used for process development as a high degree of purity can be achieved due to the wide density gradient that can be created (1.0 to 1.9 g/cm). Potassium bromide can be used at high densities, but only at elevated temperatures, i.e. 25° C., which may be incompatible with stability of some proteins. Sucrose is widely used due to being inexpensive, non-toxic and can form a gradient suitable for separation of most proteins, sub-cellular fractions and whole cells. Typically, the maximum density is about 1.3 g/cm³. The osmotic potential of sucrose can be toxic to cells in which case a complex gradient material can be used, e.g. Nycodenz. A gradient can be used with 1 or more steps in the gradient. A preferred aspect is to use a step sucrose gradient. The volume of material can is preferably from 0.5 liters to over 200 liters per run. The flow rate speed is preferably from 5 to over 25 liters per hour. The preferred operating speed is between 25,000 and 40,500 rpm producing a force of up to 122,000×g. The rotor can be unloaded statically in desired volume fractions. A preferred aspect is to unload the centrifuged material in 100 ml fractions. The isolated fraction containing the purified and concentrated lentiviral vector can then be exchanged in a desired buffer using gel filtration or size exclusion chromatography. Anionic or cationic exchange chromatography could also be used as an alternate or additional method for buffer exchange or further purification. In addition, Tangential Flow Filtration can also be used for buffer exchange and final formulation if required. Tangential Flow Filtration (TFF) can also be used as an alternative step to ultra or high-speed centrifugation, where a two-step TFF procedure would be implemented. The first step would reduce the volume of the vector supernatant, while the second step would be used for buffer exchange, final formulation and some further concentration of the material. The TFF membrane should have a membrane size of between 100 and 500 kilodaltons, where the first TFF step should have a preferable membrane size of 500 kilodaltons, while the second TFF should have a preferable membrane size of between 300 to 500 kilodaltons. The final buffer should contain materials that allow the vector to be stored for long term storage.

The present disclosure also provides methods for the concentration and purification of lentiviral vectors using either cell factories that contains adherent cells, or a bioreactor that contains suspension cells that are either transfected or transduced with the vector and accessory constructs to produce lentiviral vector. Non limiting examples of bioreactors include the Wave bioreactor system and Xcellerex bioreactors. Both are disposable systems. However non-disposable systems can also be used. The constructs can be those described herein, as well as other recombinant viral vectors. Alternatively, the cell line can be engineered to produce lentiviral vector without the need for transduction or transfection. After transfection, the lentiviral vector can be harvested and filtered to remove particulates and then is centrifuged using continuous flow high speed or ultra-centrifugation. In an aspect, a high-speed continuous flow device like the JCF-A zonal and continuous flow rotor with a high-speed centrifuge is used. Also provided is any continuous flow centrifuge where the speed of centrifugation is greater than 5,000×g RCF and less than 26,000×g RCF. Preferably, the continuous flow centrifugal force is about 10,500×g to 23.500×g RCF with a spin time of between 20 hours and 4 hours, with longer centrifugal times being used with slower centrifugal force. The lentiviral vector can be centrifuged on a cushion of more dense material (a non-limiting example is sucrose but other reagents can be used to form the cushion and these are well known in the art) so that the lentiviral vector does not form aggregates that are not filterable, as is the problem with straight centrifugation of the vector that results in a viral vector pellet. Continuous flow centrifugation onto a cushion allows the vector to avoid large aggregate formation yet allows the vector to be concentrated to high levels from large volumes of transfected material that produces the lentiviral vector. In addition, a second less-dense layer of sucrose can be used to band the lentiviral vector preparation. The flow rate for the continuous flow centrifuge is preferably between 1 and 100 ml per minute, but higher and lower flow rates can also be used. The flow rate is adjusted to provide ample time for the vector to enter the core of the centrifuge without significant amounts of vector being lost due to the high flow rate. If a higher flow rate is desired, then the material flowing out of the continuous flow centrifuge can be re-circulated and passed through the centrifuge a second time. After the virus is concentrated using continuous flow centrifugation, the vector can be further concentrated using Tangential Flow Filtration (TFF), or the TFF system can be simply used for buffer exchange. A non-limiting example of a TFF system is the Xampler cartridge system that is produced by GF>Healthcare. Preferred cartridges are those with a MW cut-off of 500.000 MW or less. Preferably a cartridge is used with a MW cut-off of 300,000 MW. A cartridge of 100,000 MW cut-off can also be used. For larger volumes, larger cartridges can be used, and those skilled in the art can find the right TFF system for this final buffer exchange and/or concentration step prior to final fill of the vector preparation. The final fill preparation may contain factors that stabilize the vector. For example, sugars are generally used and are known in the art.

### Further Cell Line Modification

In an aspect, a cell line utilized to manufacture a recombinant viral vector can be modified in any of the ways mentioned below to enhance viral vector production, e.g., by the introduction of RNAi or antisense to knock-out genes that reduce the expression of genes that limit viral vector production, or by the introduction of sequences that enhance viral vector production. Sequences that code for cellular or viral enhancers can also be engineered into cell lines (e.g., using additional plasmid vectors), such as herpes virus, hepatitis B virus, which act on HIV LTRs to enhance the level of virus product, or cellular transactivator proteins. Cellular transactivation proteins include, e.g., NF-kB, UV light responsive factors, and T cell activation factors. In another aspect, a cell line utilized to manufacture a recombinant viral vector can be modified or edited by a nuclease selected from the group consisting of a meganuclease, a zinc-finger nuclease (ZFN), a transcription activator-like effector nuclease (TALEN), a CRISPR-related nuclease (e.g., Cas9, Cas12a, etc.).

In a further aspect, a cell line utilized to manufacture a recombinant viral vector can be modified or optimized using a combination of recombination-based and gene editing-based approaches.

In an aspect, a cell line can be transformed routinely with construct DNA, e.g., using electroporation, calcium phosphate, liposomes, etc., to introduce the DNA into cells. Cells can be co-transformed (i.e., using both accessory and transfer vectors), or they can be transformed in separate steps, where each step involves the introduction of a different vector.

Cells are cultured under conditions effective to produce viral vectors. Such conditions include, e.g., the particular milieu needed to achieve protein production. Such a milieu, includes, e.g., appropriate buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including cell media, substrates, oxygen, carbon dioxide, glucose and other sugar substrates, serum, growth factors, etc.).

The present disclosure also provides for the use of cell lines that have enhanced properties for growth, reduced dependency upon expensive factors that are present in media, produce higher yields of proteins, and produce higher titers of vector particles. For example, it has recently been reported HEK 293 cells have a specific increased expression of cellular receptors and by adding the specific ligands to the medium of the cells, they demonstrated increase proliferation potential (Allison et al., Bioprocess International 2005, 3(1): 3845). A preferred aspect is a plurality of lentiviral vectors expressing an optimized combination of ligand proteins that are of relevance to HEK 293 cells after which the cells are then sorted by high throughput methods to isolate a clone of HEK 293 cells that contains multiple copies of lentiviral vectors. These cells contain a combination of HIV vectors that express different but also multiple copies of the ligand genes that are contained in the HIV vectors. The ligand genes could be codon optimized or mutations added to further increase their expression. A preferred combination is to have multiple copies of the ligand proteins expressed in the final isolated clonal cell that could then have multiple uses. It could be used for protein or antibody (including monoclonal, humanized, single-chain) production. It could also be used for the production of a vector such as a lentiviral vector, but not limited to a lentiviral vector. Other vectors such as adeno and adeno-associated vectors, murine retroviral vectors, SV40 vectors and other vectors could just as easily be produced from this now optimized cell line. A list of the receptors and their ligands that show increased expression/activity in HEK 293 cells, includes, e.g., AXL receptor (gasp); EGF receptor (EGF), chemokine receptor (fractalline); PDGF receptor, beta (PDGF); IL-15R-alpha; IL-2R-alpha; chemokine receptor 2 (MCP1); IL-2R, gamma; IL-IR-1; CSF-I receptor; oncostatin receptor; IL-4R; vitamin D3 receptor; neuropilin 1 (VEGF); macrophage stimulating receptor 1 (MSP); NGF-R; PDGFR-alpha receptor; IL-11-R, e.g., alpha; IL-10-R, e.g., beta; FGF-R-4 (aFGF); BMP receptor, e.g., type II (BMP-2); TGF-R, e.g., beta receptor II (TGF-beta); FGF-R-I (bFGF); chemokine receptor 4 (SFDIa); interferon gamma receptor 1 and 2. See, BioProcess International, 3(1), January 2005. Table 1, “Growth factor/cytokine receptors expressed by HEK-293.” Such cells will have higher protein and vector production potential and will be less dependent upon the presence of the ligand factors to be present in the medium since the cells themselves will be producing the factors and secreting them into the medium.

For other cell types, such as CHO cells, other receptor-ligand combinations may be important. For example, the insulin growth factor receptor I, insulin growth factor and insulin are thought to have anti-apoptotic activity in cells. A plurality lentiviral vectors could be constructed so that the Insulin growth factor receptor (I or II), Insulin growth factor (I or II). Insulin and the target protein for production are all contained in the vector for transduction of production cells, such as CHO cells, and an appropriate clone selected, preferably using high-throughput methods, to select the clone showing very high production of the target protein. The optimal clone may not be a cell that highly expresses all the engineered genes or inhibitors of gene expression, rather an optimal expression level of each of the genes, which for some may be a low level of expression. The value of the lentiviral vector system and using a plurality of lentiviral vectors to engineer such cell lines is that there is a random or stochastic distribution of each vector copy number in the population of cells transduced with the lentiviral vector mixture, and therefore, by varying the amount of each vector in the mixture, the number of copies of each individual second gene or inhibitory sequence can be optimized. A preferred combination of vectors and secondary gene or gene inhibitory sequences is that each lentiviral vector expresses the protein of interest for production and optionally in addition, at least one RNAi or gene that further promotes protein yield, or vector yield, either directly, or indirectly by affecting the viability or some aspect of the producing cell. However, it may also be beneficial to have at least one lentiviral vector that only expresses the secondary genes or inhibitors of gene expression in order to increase the effect of these secondary sequences.

In an aspect, the present disclosure provides for the following non-limiting embodiments:
1. A viral vector construct, wherein said construct comprises a barcoded concatemer molecule.
2. The viral vector construct of embodiment 1, wherein said barcoded concatemer molecule comprises one or more copies of an expression cassette encoding a gene of interest (GOI).
3. The viral vector construct of embodiment 2, wherein said one or more copies of said expression cassette comprises one or more unique molecular identifiers.
4. The viral vector construct of embodiment 1, wherein said one or more expression cassettes is monocistronic.
5. The viral vector construct of embodiment 3, wherein said one or more unique molecular identifiers comprises one or more spacers.
6. The viral vector construct of embodiment 3, wherein said one or more unique molecular identifiers does not comprise a spacer.
7. The viral vector construct of embodiment 3, wherein said one or more unique molecular identifiers is a single-stranded nucleic acid.
8. The viral vector construct of embodiment 3, wherein said one or more unique molecular identifiers is a double-stranded nucleic acid.
9. The viral vector construct of embodiment 3, wherein said one or more unique molecular identifiers comprise equivalent numbers of nucleotides.
10. The viral vector construct of embodiment 3, wherein said one or more unique molecular identifiers comprise different numbers of nucleotides.
11. The viral vector construct of embodiment 3, wherein said one or more unique molecular identifiers is flanked by one or more restriction enzyme sites.
12. The viral vector construct of embodiment 3, wherein said one or more unique molecular identifiers comprise a unique oligonucleotide sequence.
13. The viral vector construct of embodiment 3, wherein said one or more unique molecular identifiers comprise a known oligonucleotide sequence.
14. The viral vector construct of embodiment 12, wherein said unique oligonucleotide sequence comprises randomly incorporated oligonucleotides.
15. The viral vector construct of embodiment 1, wherein the barcoded concatemer comprises about 25 to about 50 unique molecular identifiers.
16. The viral vector construct of embodiment 3, wherein said one or more unique molecular identifiers is linked to said one or more copies of said expression cassette.
17. The viral vector construct of embodiment 16, wherein said linkage occurs via ligation.
18. The viral vector construct of embodiment 17, wherein said ligation comprises directional ligation.
19. The viral vector construct of embodiment 17, wherein said ligation comprises sequential ligation.
20. The viral vector construct of embodiment 1, wherein said barcoded concatemer molecule comprises one or more expression cassettes encoding a transcription factor.
21. The viral vector construct of embodiment 1, wherein said barcoded concatemer molecule comprises one or more expression cassettes encoding an antibiotic selection gene.
22. The viral vector construct of embodiment 1, wherein said barcoded concatemer molecule comprises one or more insulator sequences.
23. The viral vector construct of embodiment 1, wherein said barcoded concatemer molecule comprises two or more restriction enzyme sites.
24. The viral vector construct of embodiment 1, wherein said barcoded concatemer molecule comprises one or more elements selected from the group consisting of a 5′ long terminal repeat, a 3′ long terminal repeat, a packaging signal, and a central polypurine tract.
25. The viral vector construct of embodiment 1, wherein said barcoded concatemer molecule does not comprise a 5′ long terminal repeat, a 3′ long terminal repeat, a packaging signal, or a central polypurine tract.
26. The barcoded concatemer molecule of embodiment 24, wherein said 5′ long terminal repeat is chimeric.
27. The viral vector construct of embodiment 1, wherein said viral vector construct is target-specific.
28. The viral vector construct of embodiment 1, wherein said viral vector construct is derived from a retrovirus.
29. The viral vector construct of embodiment 1, wherein said viral vector construct is derived from a lentivirus.
30. The viral vector construct of embodiment 1, wherein said viral vector construct is derived from a herpesvirus.
31. The viral vector construct of embodiment 1, wherein said viral vector construct is derived from an adenovirus.
32. The viral vector construct of embodiment 1, wherein said viral vector construct is derived from an adeno-associated virus.
33. The viral vector construct of embodiment 1, wherein said viral vector construct comprises a self-inactivating long terminal repeat.
34. The viral vector construct of embodiment 1, wherein said viral vector construct comprises one or more selectable or reporter elements.
35. The viral vector construct of embodiment 34, wherein said one or more selectable or reporter elements is a reporter gene, an epitope tag, or both.
36. The viral vector construct of embodiment 35, wherein said one or more selectable or reporter elements is selected or detected by luminescence, absorbance, fluorescence.
antibiotics, antigen-antibody interactions, or a combination thereof.
37. The viral vector construct of embodiment 1, wherein said viral vector construct comprises a promoter and a polyadenylation sequence.
38. The viral vector construct of embodiment 37, wherein said promoter is constitutive or inducible.
39. The viral vector construct of embodiment 38, wherein said inducible promoter comprises a tetracycline-dependent promoter.
40. The viral vector construct of embodiment 37, wherein said promoter is synthetic.
41. A cell line comprising a viral vector construct comprising a barcoded concatemer molecule.
42. The cell line of embodiment 41, wherein said cell line is in a cell culture comprising a volume of medium.
43. The cell line of embodiment 41, wherein said cell line is adapted for adherent culturing or culturing in suspension.
44. The cell line of embodiment 41, wherein said cell line is cultured in a serum-supplemented or a serum-free medium.
45. The cell line of embodiment 41, wherein said cell line is immortalized.
46. The cell line of embodiment 41, wherein said cell line is eukaryotic.
47. The cell line of embodiment 41, wherein said cell line is mammalian.
48. The cell line of embodiment 41, wherein said cell line is human.
49. The cell line of embodiment 41, wherein said cell line is a HEK293 cell line or a derivative thereof.
50. The cell line of embodiment 49, wherein said HEK293 cell line is a HEK293T cell line.
51. The cell line of embodiment 41, wherein said cell line produces psuedotyped viral particles.
52. The cell line of embodiment 51, wherein said psuedotyped viral particles comprise one or more envelope proteins of a virus selected from the group consisting of Vesiculovirus, Gammaretrovirus, and Morbillivirus.
53. The cell line of embodiment 41, wherein said cell line produces mosaic viral particles.
54. The cell line of embodiment 41, wherein said cell line produces chimeric viral particles.
55. The cell line of embodiment 41, wherein said barcoded concatemer molecule comprises one or more expression cassettes encoding a gene of interest (GOI) and one or more unique molecular identifiers.
56. The cell line of embodiment 55, wherein said one or more expression cassettes is monocistronic.
57. The cell line of embodiment 55, wherein said unique molecular identifier is a single-stranded nucleic acid.
58. The cell line of embodiment 55, wherein said unique molecular identifier is a double-stranded nucleic acid.
59. The cell line of embodiment 55, wherein said one or more unique molecular identifiers comprises one or more spacers.
60. The cell line of embodiment 59, wherein said one or more unique molecular identifiers does not comprise a spacer.
61. The cell line of embodiment 59, wherein said one or more unique molecular identifiers comprise equivalent numbers of nucleotides.
62. The cell line of embodiment 59, wherein said one or more unique molecular identifiers comprise different numbers of nucleotides.
63. The cell line of embodiment 59, wherein said one or more unique molecular identifiers is flanked by one or more restriction enzyme sites.
64. The cell line of embodiment 59, wherein the barcoded concatemer comprises about 25 to about 50 unique molecular identifiers.
65. The cell line of embodiment 59, wherein said unique molecular identifier is linked to said expression cassette.
66. The cell line of embodiment 65, wherein said linkage occurs via ligation.
67. The cell line of embodiment 66, wherein said ligation comprises directional ligation.
68. The cell line of embodiment 66, wherein said ligation comprises sequential ligation.
69. The cell line of embodiment 41, wherein said barcoded concatemer molecule comprises one or more expression cassettes encoding a transcription factor.
70. The cell line of embodiment 41, wherein said barcoded concatemer molecule comprises one or more expression cassettes encoding an antibiotic selection gene.
71. The cell line of embodiment 41, wherein said barcoded concatemer molecule comprises one or more insulator sequences.
72. The cell line of embodiment 41, wherein said barcoded concatemer molecule comprises two or more restriction enzyme sites.
73. The cell line of embodiment 41, wherein said barcoded concatemer molecule comprises one or more elements selected from the group consisting of a 5′ long terminal repeat, a 3′ long terminal repeat, a packaging signal, and a central polypurine tract.
74. The cell line of embodiment 41, wherein said barcoded concatemer molecule does not comprise a 5′ long terminal repeat, a 3′ long terminal repeat, a packaging signal, or a central polypurine tract.
75. The cell line of embodiment 73, wherein said 5′ long terminal repeat is chimeric.
76. The cell line of embodiment 41, wherein said viral vector construct is target-specific.
77. The cell line of embodiment 41, wherein said viral vector construct is derived from a retrovirus.
78. The cell line of embodiment 41, wherein said viral vector construct is derived from a lentivirus.
79. The cell line of embodiment 41, wherein said viral vector construct is derived from a herpesvirus.
80. The cell line of embodiment 41, wherein said viral vector construct is derived from an adenovirus.
81. The cell line of embodiment 41, wherein said viral vector construct is derived from an adeno-associated virus.
82. The cell line of embodiment 41, wherein said viral vector construct comprises a self-inactivating long terminal repeat.
83. The cell line of embodiment 41, wherein said viral vector construct comprises one or more selectable or reporter elements.
84. The viral vector construct of embodiment 83, wherein said one or more selectable or reporter elements is a reporter gene, an epitope tag, or both.
85. The viral vector construct of embodiment 84, wherein said one or more selectable or reporter elements is selected or detected by luminescence, absorbance, fluorescence, antibiotics, antigen-antibody interactions, or a combination thereof.
86. The cell line of embodiment 41, wherein said viral vector construct comprises a promoter and a polyadenylation sequence.
87. The viral vector construct of embodiment 86, wherein said promoter is constitutive or inducible.
88. The viral vector construct of embodiment 87, wherein said inducible promoter comprises a tetracycline-dependent promoter.
89. The viral vector construct of embodiment 87, wherein said promoter is synthetic.
90. A method of making a viral vector construct comprising a barcoded concatemer molecule, said method comprising:
   a. ligating a unique molecular identifier to a viral vector genome construct to generate a barcoded viral vector genome construct; and
   b. ligating said barcoded viral vector genome construct to generate said barcoded concatemer molecule.
91. The method of embodiment 90, wherein said viral vector construct comprises one or more unique molecular identifiers.
92. The method of embodiment 90, wherein said viral vector construct comprises about 25 to about 50 unique molecular identifiers.
93. The method of embodiment 90, wherein said unique molecular identifier is flanked by one or more restriction enzyme sites.
94. The method of embodiment 93, wherein said one or more restriction enzyme sites is located at a 5′ end of said unique molecular identifier.
95. The method of embodiment 93, wherein said one or more restriction enzyme sites is located at a 3′ end of said unique molecular identifier.
96. The method of embodiment 90, wherein said viral vector genome construct comprises one or more restriction enzyme sites.
97. The method of embodiment 90, wherein said unique molecular identifier is ligated to said viral vector genome construct prior to concatemer ligation.
98. The method of embodiment 90, wherein said ligation of said unique molecular identifier to said viral vector genome construct occurs via beads.
99. The method of embodiment 90, wherein said beads comprise magnetic beads.
100. The method of embodiment 90, wherein said ligation of said unique molecular identifier to said viral vector genome construct occurs without use of beads.
101. The method of embodiment 98, wherein said beads comprise one or more streptavidin molecules.
102. The method of embodiment 90, wherein said unique molecular identifier is biotinylated.
103. The method of embodiment 102, wherein said unique molecular identifier is biotinylated at a 5′ end.
104. The method of embodiment 90, wherein said unique molecular identifier is a single-stranded nucleic acid.
105. The method of embodiment 90, wherein said unique molecular identifier is a double-stranded nucleic acid.
106. A method of making a stable viral vector producer cell line comprising a barcoded concatemer, said method comprising:
   a) introducing into a population of cells a viral vector genome construct, wherein said viral vector genome construct comprises a barcoded concatemer, comprising an expression cassette encoding one or more copies of a gene of interest (GOI) and one or more unique molecular identifiers;
   b) producing a population of transgenic cells comprising integrated sequences encoding said GOI and said unique molecular identifiers;
   c) selecting from said population of transgenic cells a cell clone producing a desired viral titer; and
   d) generating from said cell clone a stable viral vector producer cell line.
107. The method of embodiment 106, wherein said transgenic cells comprise polyclonal cells.
108. The method of embodiment 106, wherein said selecting further comprises polyclonal to monoclonal selection of said transgenic cells.
109. The method of embodiment 108, wherein said polyclonal to monoclonal selection comprises limiting dilution, single cell sorting, or both.
110. The method of any one of embodiments 106 to 109, wherein said generation of said stable vector producer cell line occurs by expansion of said selected cell clone.
111. The method of embodiment 106, wherein said method further comprises storing said selected cell line by cryopreservation.
112. The method of embodiment 111, wherein said method further comprises expanding cells from said cryopreserved cell line to produce viral vectors.
113. The method of embodiment 106, wherein said method further comprises quantifying the level of said viral vector genome in said selected cell clone, said generated cell line, or both.
114. The method of embodiment 106, wherein said method further comprises harvesting viral vector from said selected cell clone, said generated cell line or both.
115. The method of embodiment 106, wherein said method further comprises determining a viral titer of said selected cell clone, said generated cell line, or both.
116. The method of embodiment 115, wherein determining said viral titer comprises physical titration, functional titration, or both.
117. The method of embodiment 115, wherein determining said viral titer comprises PCR, immunodetection, ELISA, or any combination thereof.
118. The method of embodiment 116, wherein said method further comprises determining an infectivity of said viral titer of said selected cell clone or said generated cell line.
119. The method of embodiment 106, wherein said viral vector producer cell line produces a target-specific viral vector.
120. The method of embodiment 106, wherein said viral vector producer cell line produces a viral vector derived from a retrovirus.
121. The method of embodiment 106, wherein said viral vector producer cell line produces a viral vector derived from a lentivirus.
122. The method of embodiment 106, wherein said viral vector producer cell line produces a viral vector derived from a herpesvirus.
123. The method of embodiment 106, wherein said viral vector producer cell line produces a viral vector derived from an adenovirus.
124. The method of embodiment 106, wherein said viral vector producer cell line produces a viral vector derived from an adeno-associated virus.
125. The method of embodiment 106, wherein said viral vector producer cell line produces a viral vector comprising one or more capsid proteins.
126. The method of embodiment 125, wherein said one or more capsid proteins are heterologous.
127. The method of embodiment 125, wherein said one or more capsid proteins are genetically modified.
128. The method of embodiment 125, wherein said one or more capsid proteins are chemically modified.
129. The method of embodiment 106, wherein said viral vector producer cell line produces a viral vector comprising one or more envelope proteins.
130. The method of embodiment 129, wherein said one or more envelope proteins are heterologous.
131. The method of embodiment 106, wherein said viral vector genome construct comprises 5′ and 3′ long terminal repeats, a packaging signal, and a central polypurine tract.
132. The method of embodiment 131, wherein said 5′ long terminal repeat is chimeric.
133. The method of embodiment 106, wherein said viral vector genome construct comprises a self-inactivating long terminal repeat.
134. The method of embodiment 106, wherein said viral vector genome construct comprises one or more selectable or detectable elements.
135. The method of embodiment 134, wherein said one or more selectable or detectable elements is a reporter gene, an epitope tag, or both.
136. The method of embodiment 134, wherein said one or more selectable or detectable elements is selected or detected by luminescence, absorbance, fluorescence, antibiotics, antigen-antibody interactions, or a combination thereof.
137. The method of embodiment 106, wherein said viral vector genome construct comprises a promoter and a polyadenylation sequence.
138. The method of embodiment 137, wherein said promotor of said viral vector genome construct is constitutive or inducible.
139. The method of embodiment 137, wherein said promotor of said viral vector genome construct is synthetic.
140. The method of embodiment 106, wherein said concatemer comprises multiple copies of an expression cassette encoding said GOI.
141. The method of embodiment 106, wherein said concatemer comprises one or more expression cassettes encoding a transcription factor.
142. The method of embodiment 106, wherein said introducing step comprises a chemical, biological, or physical step.
143. The method of embodiment 106, wherein said introducing step comprises an optical method, a magnetic method, a biolistic method, a polymer-based method, a liposome-based method, a nanoparticle-based method, or a combination thereof.
144. The method of embodiment 106, wherein said introducing step comprises transduction.
145. The method of embodiment 106, wherein said introducing step comprises transfection.
146. The method of embodiment 142, wherein said chemical introducing step comprises the use of a cationic polymer, calcium phosphate, cationic lipid, or a combination thereof.
147. The method of embodiment 142, wherein said biological introducing step comprises introduction via a retrovirus, lentivirus, transposon, TALEN, Zinc Finger nuclease, meganuclease, transposase, CRISPR-related nuclease, or recombinase.
148. The method of embodiment 147, wherein said recombinase comprises Cre-recombinase or Flippase recombinase.
149. The method of embodiment 142, wherein said physical introducing step is selected from the group consisting of electroporation, sonoporation, mechanoporation, and photoporation.
150. The method of embodiment 106, wherein said integrated sequences exhibit random integration.
151. The method of embodiment 106, wherein said integrated sequences exhibit site-specific integration.
152. The method of embodiment 106, wherein said stable viral vector producer cell line is in a cell culture that comprises a volume of medium.
153. The method of embodiment 106, wherein said stable viral vector producer cell line is adapted for adherent culturing or culturing in suspension.
154. The method of embodiment 106, wherein said stable viral vector producer cell line is cultured in a serum-supplemented or a serum-free medium.
155. The method of embodiment 106, wherein said stable viral vector producer cell line is immortalized.
156. The method of embodiment 106, wherein said stable viral vector producer cell line is eukaryotic.
157. The method of embodiment 106, wherein said stable viral vector producer cell line is mammalian.
158. The method of embodiment 106, wherein said stable viral vector producer cell line is human.
159. The method of embodiment 106, wherein said stable viral vector producer cell line is a HEK293 cell or a derivative thereof
160. The method of embodiment 159, wherein said HEK293 cell line is a HEK293 T cell line.
161. The method of embodiment 106, wherein said stable viral vector producer cell line produces psuedotyped viral particles.
162. The method of embodiment 161, wherein said psuedotyped viral particles comprise one or more envelope proteins of a virus selected from the group consisting of Vesiculovirus. Gammaretrovirus, and Morbillivirus.
163. The method of embodiment 106, wherein said stable viral vector producer cell line produces mosaic viral particles.
164. The method of embodiment 106, wherein said stable viral vector producer cell line produces chimeric viral particles.
165. A method of characterizing a viral vector comprising a barcoded concatemer molecule, wherein said viral vector is integrated, and wherein said barcoded concatemer comprises one or more unique molecular identifiers, said method comprising:
   a. sequencing the barcoded concatemer molecule to generate sequence reads;
   b. assembling said generated sequence reads; and
   c. mapping said assembled sequence reads.
166. The method of embodiment 165, wherein said assembling of said generated sequence reads enables quantifying a number of viral vector genome constructs comprised in said barcoded concatemer.
167. The method of embodiment 165, wherein said mapping of said assembled sequence reads enables identification of a genomic location of an integrated viral vector comprising said barcoded concatemer molecule.
168. The method of embodiment 165, wherein said method comprises generating sequencing reads that comprise one or more unique molecular identifier sequences.
169. The method of embodiment 165, wherein said method comprises generating sequencing reads that comprise one or more vector genome sequences and one or more cell genomic sequences.
170. The method of embodiment 165, wherein said method comprises generating sequencing reads that comprise one or more vector genome sequences.
171. The method of embodiment 168, wherein said sequencing reads comprising said one or more unique molecular identifier sequences facilitates quantification of a number of viral vector genome constructs comprised in a concatemer.
172. The method of embodiment 169, wherein said sequencing reads comprising said one or more vector genome sequences and said one or more cell genomic sequences facilitates determination of a locus of integration of said barcoded concatemer molecule.
173. The method of embodiment 165, wherein said method comprises quantifying one or more selectable or reporter elements of a viral vector construct.
174. The method of embodiment 173, wherein said one or more selectable or reporter elements is a reporter gene, an epitope tag, or both.
175. The method of embodiment 173, wherein said one or more selectable or reporter elements is selected or detected by luminescence, absorbance, fluorescence, antibiotics, antigen-antibody interactions, or a combination thereof.
176. The method of embodiment 166, wherein said quantifying comprises quantifying a number of vector-vector junctions.
177. The method of embodiment 166, wherein said quantifying comprises quantifying a number of vector-cellular genome junctions.
178. The method of embodiment 166, wherein said quantifying comprises quantifying a number of vector-transgene junctions.
179. The method of embodiment 178, wherein said transgene comprises a gene of interest (GOI).

Having now generally described the disclosure, the same will be more readily understood through reference to the following examples that are provided by way of illustration and are not intended to be limiting of the present disclosure, unless specified.

### EXAMPLES

### Example 1

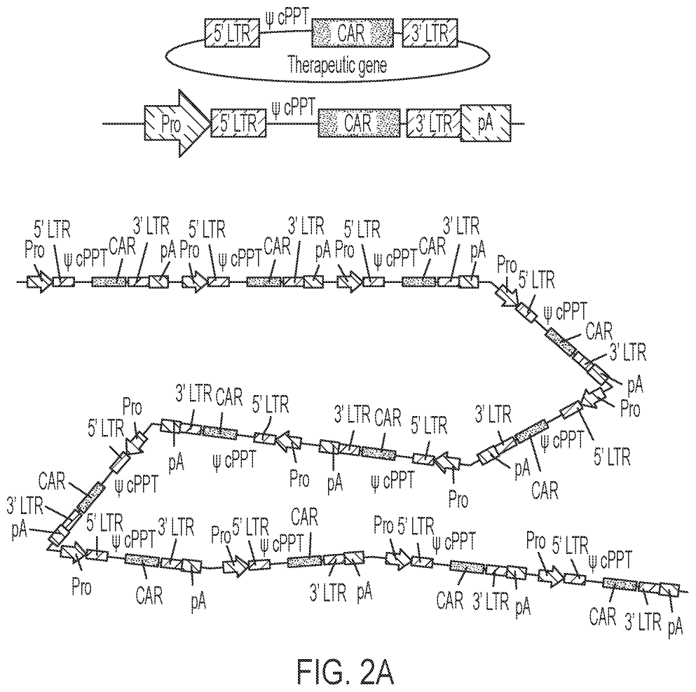

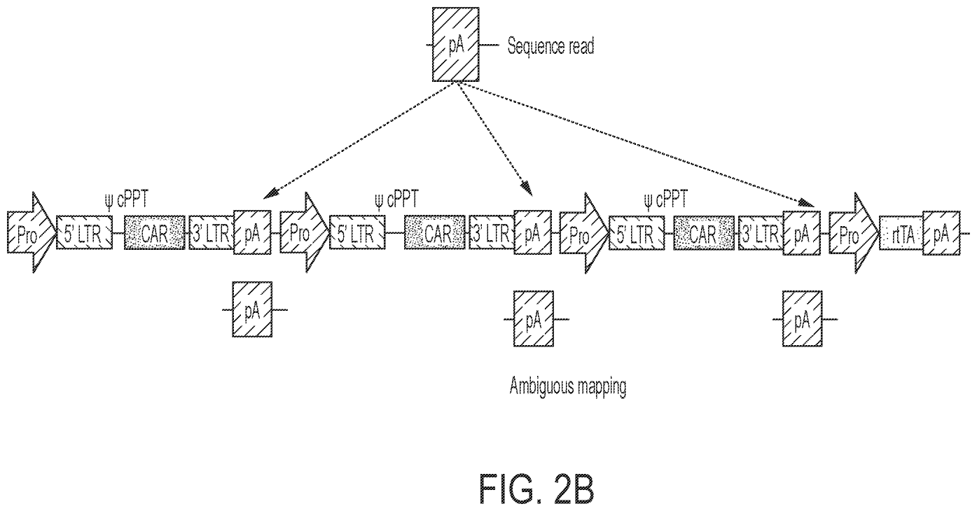

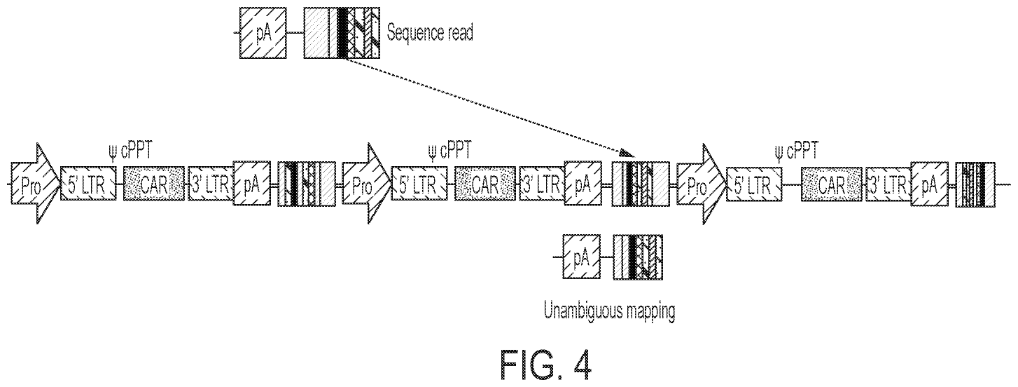

As an illustration of the concept described here, in a non-barcoded concatemer, a vector genome construct is directionally ligated (head-to-tail) to itself to form long chains (e.g., FIG. 2A). As this concatemer (ligated chain) is essentially a long stretch of substantially similar sequences, it functionally behaves as a repetitive element. While generating sequence reads via genomic sequencing is largely unaffected by this construction, the ability to map sequence reads to a location is hindered, unless a unique sequence is present. Many of the sequence reads are considered ambiguous for assembling and mapping purposes because many reads are short, for example, 100-200 base pairs, and the repeating vector element longer, for example 3-10 kb (e.g., FIG. 2B). For example, if a vector genome sequence is ABCD and the actual inserted concatemer sequence is XABCDABCDABCDZ (where X and Z are unique flaking genomic sequences) and the reads are XA, AB, BC. CD, DA, and DX, one would not know whether the true sequence is XABCDABCDZ or XABCDABCDABCDABCDABCDX. The barcoded concatemer of the present disclosure changes this ABCD to ABCD+Barcode (e.g., FIG. 4). In this example, a barcode is abbreviated with lowercase letters. In a barcoded concatemer, the actual integrated construct is, for example, XABCDaABCDbABCDcABCDdZ, and the reads are, for example, XA, AB, BC, CD, DaA, DbA, DcA, DdX. In this example, some of the reads are still ambiguous (AB, BC, and CD), but others facilitate an accurate accounting of the number of repeats, which in this example is four, not including the 5′ and 3′ ends: XA=5′ end, DaA=1, DbA=2, DcA=3, DdX=4, and the 3′ end. The present disclosure thereby enables discernment of a genomic location of a concatemer insert and the number of viral vector genomes contained in a given concatemer, which are useful data points, for example, in quality and regulatory contexts.

### Example 2

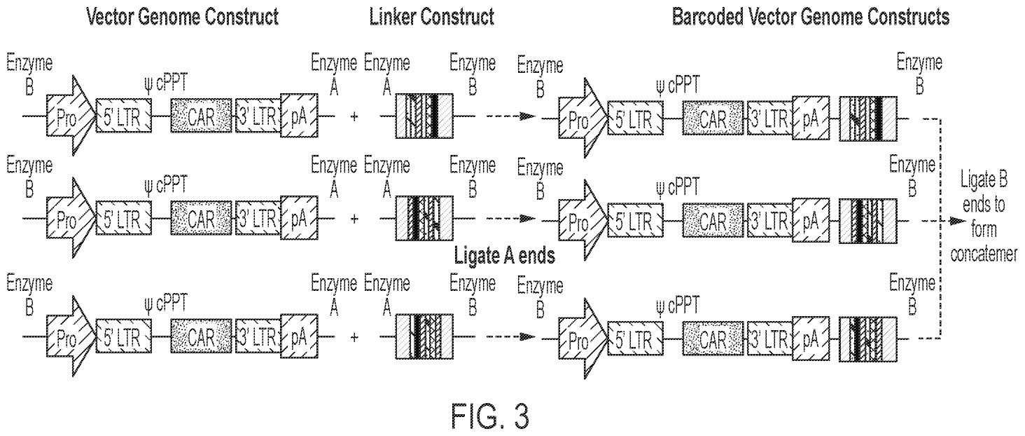

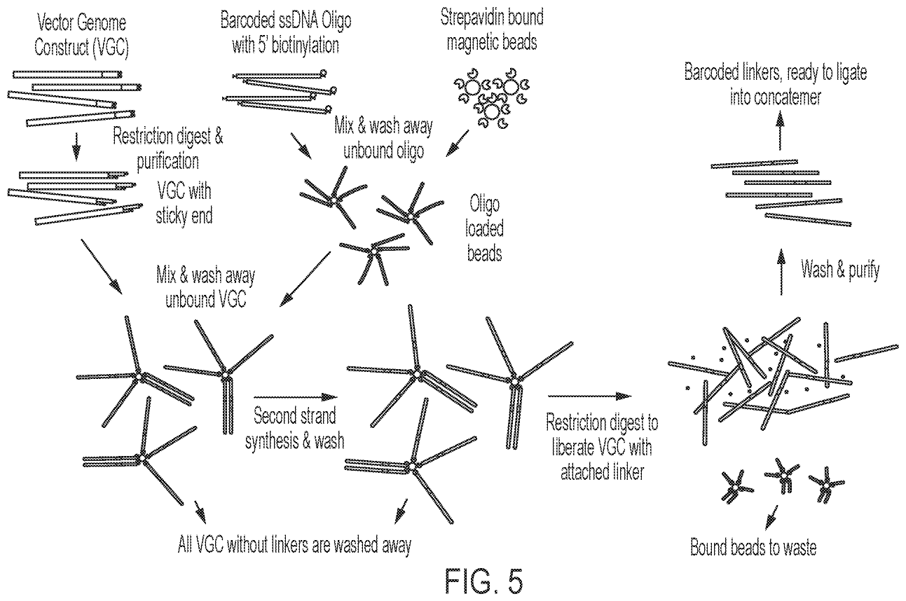

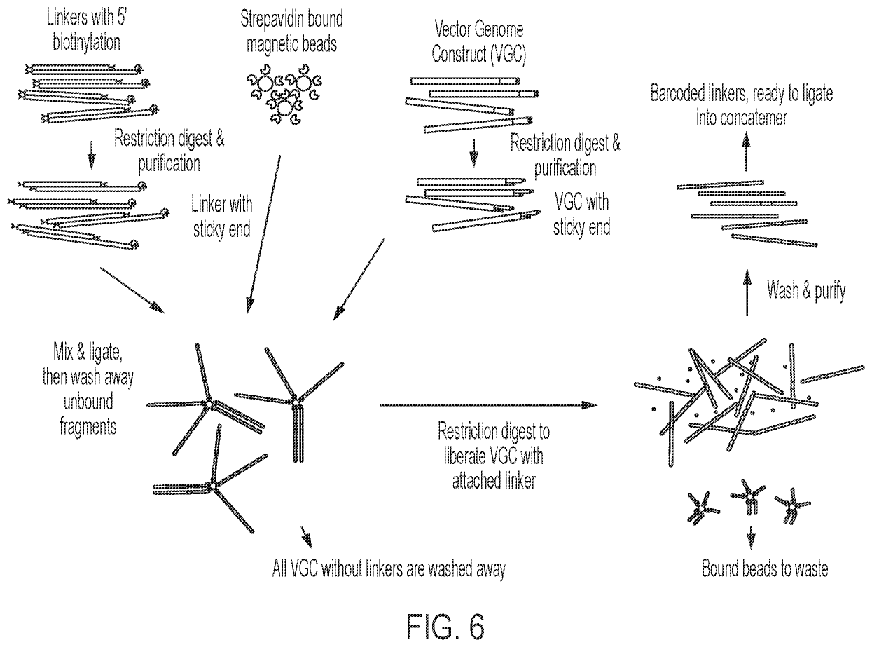

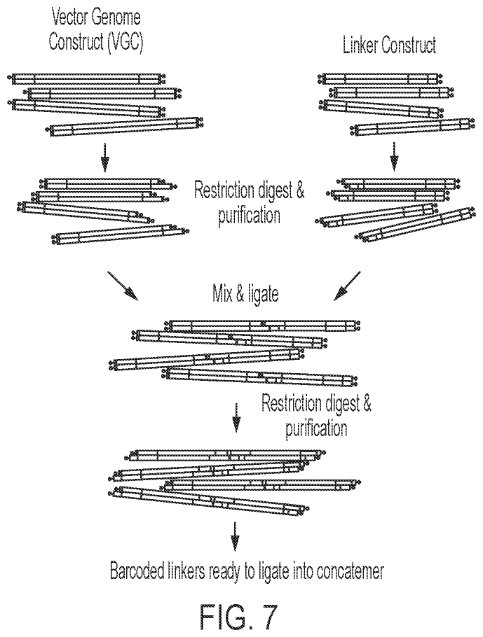

As an illustration of the concept described here, a barcode is embedded in an linker that is flanked by an upstream and downstream restriction enzyme sites (site A and site B, recognized and cut by enzyme A and enzyme B, respectively) (a unique molecular identifier) (e.g., FIG. 3). Linkers are synthesized with a number of randomly incorporated nucleotides to create a diverse library of unique sequences flanked by known sequences. Restriction enzyme sites are designed into the linkers, cut with restriction enzymes to generate, for example, sticky ends. The sticky ends are used to directionally sequentially ligate vector to linker and vector-linker to concatemer. Enzyme A cuts the vector genome sequence at its 3′ end, for example, and the linker at its 5′ end, for example. A vector genome sequence cut at its 3′ end is ligated with a linker cut at its 5′ end, for example, to create a vector+linker fragment. A vector-linker fragment is digested by restriction enzyme B to cut the vector genome at its 5′ end, and the linker at its 3′ end to create a B sticky end+vector+linker+B sticky end fragment. These are then ligated into a concatemer (e.g., FIGS. 5-7). Concatemer ligation products can be selected to ensure that unincorporated fragments are removed are removed from a transfection mix. Ligation products can be selected, for example, by size, such that smaller unincorporated fragments are removed from a transfection mix.

### Example 3

As an illustration of the concept described here, following genomic sequencing, performed by methods known in the art, multiple types of sequence reads are generated (e.g., FIG. 4). Exemplary types of sequence reads include: reads that contain a barcode sequence (with or without linked vector genome sequences), reads that contain vector genome sequences with linked cell genomic sequences, and reads that contain only vector genome sequences. Types of sequencing reads vary with respect to the utility of information provided for subsequent steps of sequence assembly and sequence mapping. For example, reads containing a barcode sequence (with or without linked vector genome sequences) cannot be assigned to a specific location, but the number of unique barcodes recovered equals the number of vector genomes in the concatemer. Reads containing vector genome sequences with linked cell genomic sequences are informative as they describe the location of the concatemer in the producer cell genome. Reads containing only vector genome sequences may be less informative than reads described in the prior two sentences. Thus, provided in this disclosure is a mechanism that facilitates determination of an insertion site of an integrated concatemer and a number of copies of vector genome in a barcoded concatemer. The embedded linkers and barcodes (unique molecular identifiers) enable interpretation of raw sequence data, and therefore, the disclosed method is compatible with many sequencing platforms, as specific primer binding is not required.

1. A viral vector construct, wherein said construct comprises a barcoded concatemer molecule comprising one or more copies of an expression cassette encoding a gene of interest (GOI).

2. (canceled)

3. The viral vector construct of claim 1, wherein said one or more copies of said expression cassette comprises one or more unique molecular identifiers.

4.-10. (canceled)

11. The viral vector construct of claim 3, wherein said one or more unique molecular identifiers is flanked by one or more restriction enzyme sites.

12.-23. (canceled)

24. The viral vector construct of claim 1, wherein said barcoded concatemer molecule comprises one or more elements selected from the group consisting of a 5′ long terminal repeat, a 3′ long terminal repeat, a packaging signal, and a central polypurine tract.

25. (canceled)

26. (canceled)

27. (canceled)

28. The viral vector construct of claim 1, wherein said viral vector construct is derived from a retrovirus, a herpesvirus, an adenovirus, or an adeno-associated virus.

29. The viral vector construct of claim 1, wherein said viral vector construct is derived from a lentivirus.

30. (canceled)

31. (canceled)

32. (canceled)

33. The viral vector construct of claim 1, wherein said viral vector construct comprises a self-inactivating long terminal repeat.

34. The viral vector construct of claim 1, wherein said viral vector construct comprises one or more selectable or reporter elements.

35.-89. (canceled)

90. A method of making a viral vector construct comprising a barcoded concatemer molecule, said method comprising:
a. ligating a unique molecular identifier to a viral vector genome construct to generate a barcoded viral vector genome construct; and
b. ligating a concatemer molecule to said barcoded viral vector genome construct to generate said barcoded concatemer molecule.

91. (canceled)

92. (canceled)

93. The method of claim 90, wherein said unique molecular identifier is flanked by one or more restriction enzyme sites.

94. (canceled)

95. (canceled)

96. (canceled)

97. (canceled)

98. The method of claim 90, wherein said ligation of said unique molecular identifier to said viral vector genome construct occurs via an intermediary comprising beads, magnetic beads, or beads comprising one or more streptavidin molecules.

99. (canceled)

100. (canceled)

101. (canceled)

102. The method of claim 90, wherein said unique molecular identifier is biotinylated.

103. (canceled)

104. (canceled)

105. (canceled)

106. A method of making a stable viral vector producer cell line comprising a barcoded concatemer, said method comprising:
a) introducing into a population of cells a viral vector genome construct, wherein said viral vector genome construct comprises a barcoded concatemer, comprising an expression cassette encoding one or more copies of a gene of interest (GOI) and one or more unique molecular identifiers;
b) producing a population of transgenic cells comprising integrated sequences encoding said GOI and said unique molecular identifiers;
c) selecting from said population of transgenic cells a cell clone producing a desired viral titer; and
d) generating from said cell clone a stable viral vector producer cell line.

107.-118. (canceled)

119. The method of claim 106, wherein said viral vector producer cell line produces a target-specific viral vector.

120. (canceled)

121. The method of claim 106, wherein said viral vector producer cell line produces a viral vector derived from a lentivirus.

122. (canceled)

123. (canceled)

124. The method of claim 106, wherein said viral vector producer cell line produces a viral vector derived from a retrovirus, a herpesvirus, adenovirus, or an adeno-associated virus.

125. The method of claim 106, wherein said viral vector producer cell line produces a viral vector comprising one or more capsid proteins or one or more envelope proteins.

126.-130. (canceled)

131. The method of claim 106, wherein said viral vector genome construct comprises 5′ and 3′ long terminal repeats, a packaging signal, and a central polypurine tract.

132. (canceled)

133. (canceled)

134. The method of claim 106, wherein said viral vector genome construct comprises one or more selectable or detectable elements.

135.-164. (canceled)

165. A method of characterizing a viral vector comprising a barcoded concatemer molecule, wherein said viral vector is integrated, and wherein said barcoded concatemer comprises one or more unique molecular identifiers, said method comprising:
a. sequencing the barcoded concatemer molecule to generate sequence reads;
b. assembling said generated sequence reads; and
c. mapping said assembled sequence reads.

166.-179. (canceled)
